SEQUENCE LISTING (62 sequences)

SEQ ID NO: 1 - DNA, 786 bp, Mycobacterium tuberculosis
atgcccaagagaagcgaatacaggcaaggcacgccgaactgggtcgaccttcagaccaccgatcagtccgccgccaaaaagttctacacatcgttgttcggctggggttacgacgacaacccggtccccggaggcggtggggtctattccatggccacgctgaacggcgaagccgtggccgccatcgcaccgatgcccccgggtgcaccggaggggatgccgccgatctggaacacctatatcgcggtggacgacgtcgatgcggtggtggacaaggtggtgcccgggggcgggcaggtgatgatgccggccttcgacatcggcgatgccggccggatgtcgttcatcaccgatccgaccggcgctgccgtgggcctatggcaggccaatcggcacatcggagcgacgttggtcaacgagacgggcacgctcatctggaacgaactgctcacggacaagccggatttggcgctagcgttctacgaggctgtggttggcctcacccactcgagcatggagatagctgcgggccagaactatcgggtgctcaaggccggcgacgcggaagtcggcggctgtatggaaccgccgatgcccggcgtgccgaatcattggcacgtctactttgcggtggatgacgccgacgccacggcggccaaagccgccgcagcgggcggccaggtcattgcggaaccggctgacattccgtcggtgggccggttcgccgtgttgtccgatccgcagggcgcgatcttcagtgtgttgaagcccgcaccgcagcaatag

SEQ ID NO: 2 - protein, 261 aa, Mycobacterium tuberculosis
MPKRSEYRQGTPNWVDLQTTDQSAAKKFYTSLFGWGYDDNPVPGGGGVYSMATLNGEAVAAIAPMPPGAPEGMPPIWNTYIAVDDVDAVVDKVVPGGGQVMMPAFDIGDAGRMSFITDPTGAAVGLWQANRHIGATLVNETGTLIWNELLTDKPDLALAFYEAVVGLTHSSMEIAAGQNYRVLKAGDAEVGGCMEPPMPGVPNHWHVYFAVDDADATAAKAAAAGGQVIAEPADIPSVGRFAVLSDPQGAIFSVLKPAPQQ

SEQ ID NO: 3 - DNA, 33 bp, Artificial Sequence
  Description of Artificial Sequence  Primer
attaaagcttacccgaccgcgtgaccagcggtc

SEQ ID NO: 4 - DNA, 33 bp, Artificial Sequence
  Description of Artificial Sequence  Primer
attatctagagatcatcctttcgttaggtggcg

SEQ ID NO: 5 - DNA, 32 bp, Artificial Sequence
  Description of Artificial Sequence  Primer
attatctagacagcaatagggagcatcccggg

SEQ ID NO: 6 - DNA, 26 bp, Artificial Sequence
  Description of Artificial Sequence  Primer
attagcagcgacggtgtcaacggttc

SEQ ID NO: 7 - DNA, 30 bp, Artificial Sequence
  Description of Artificial Sequence  Primer
attatctagaccctgtgaatagaggtccgc

SEQ ID NO: 8 - DNA, 30 bp, Artificial Sequence
  Description of Artificial Sequence  Primer
atttctagactggaggagatgatcgaggat

SEQ ID NO: 9 - DNA, 27 bp, Artificial Sequence
  Description of Artificial Sequence  Primer
atattaattaagatgcccaagagaagc

SEQ ID NO: 10 - DNA, 26 bp, Artificial Sequence
  Description of Artificial Sequence  Primer
attggatccctattgctgcggtgcgg

SEQ ID NO: 11 - DNA, 33 bp, Artificial Sequence
  Description of Artificial Sequence  Primer
ggactagtgcgatcagccagtcgatcagcagag

SEQ ID NO: 12 - DNA, 34 bp, Artificial Sequence
  Description of Artificial Sequence  Primer
ccttaattaattccgtcacagaatgctcctttac

SEQ ID NO: 13 - DNA, 28 bp, Artificial Sequence
  Description of Artificial Sequence  Primer
cccttaaatgtccgccacctaacgaaag

SEQ ID NO: 14 - DNA, 24 bp, Artificial Sequence
  Description of Artificial Sequence  Primer
cccaagcttctagcattctccgaa

SEQ ID NO: 15 - DNA, 25 bp, Artificial Sequence
  Description of Artificial Sequence  Primer
acggtgggtactaggtgtgggtttc

SEQ ID NO: 16 - DNA, 28 bp, Artificial Sequence
  Description of Artificial Sequence  Primer
tctgcgattactagcgactccgacttca

SEQ ID NO: 17 - DNA, 18 bp, Artificial Sequence
  Description of Artificial Sequence  Primer
ggcgatctggtgggcccg

SEQ ID NO: 18 - DNA, 21 bp, Artificial Sequence
  Description of Artificial Sequence  Primer
cgccggaggcattagcacgct

SEQ ID NO: 19 - DNA, 19 bp, Artificial Sequence
  Description of Artificial Sequence  Primer
acgcgaagtcgggcaacac

SEQ ID NO: 20 - DNA, 22 bp, Artificial Sequence
  Description of Artificial Sequence  Primer
gcggcagcgggcgggagcaact

SEQ ID NO: 21 - DNA, 27 bp, Artificial Sequence
  Description of Artificial Sequence  Primer
atgcccaagagaagcgaatacaggcaa

SEQ ID NO: 22 - DNA, 24 bp, Artificial Sequence
  Description of Artificial Sequence  Primer
ctattgctgcggtgcgggcttcaa

SEQ ID NO: 23 - DNA, 24 bp, Artificial Sequence
  Description of Artificial Sequence  Primer
cgaggggattacacatgaccaact

SEQ ID NO: 24 - DNA, 19 bp, Artificial Sequence
  Description of Artificial Sequence  Primer
cggaagagcgcccaatacg

SEQ ID NO: 25 - DNA, 24 bp, Artificial Sequence
  Description of Artificial Sequence  Primer
ataacgttctcggctcgatgatcc

SEQ ID NO: 26 - DNA, 29 bp, Artificial Sequence
  Description of Artificial Sequence  Primer
atcccctgattctgtggataaccgtatta

SEQ ID NO: 27 - DNA, 22 bp, Artificial Sequence
  Description of Artificial Sequence  Primer
accaccttgtccaccaccgcat

SEQ ID NO: 28 - DNA, 24 bp, Artificial Sequence
  Description of Artificial Sequence  Primer
cgaatcattggcacgtctactttg

SEQ ID NO: 29 - DNA, 23 bp, Artificial Sequence
  Description of Artificial Sequence  Primer
gtggcttggcgggcacggtggag

SEQ ID NO: 30 - DNA, 25 bp, Artificial Sequence
  Description of Artificial Sequence  Primer
gtggcaccggcggcaccgcacacct

SEQ ID NO: 31 - DNA, 75 bp, Mycobacterium tuberculosis
atgcccaagagaagcgaatacaggcaaggcacgccgaactgggtcgaccttcagaccaccgatcagtccgccgcc

SEQ ID NO: 32 - protein, 25 aa, Mycobacterium tuberculosis
MPKRSEYRQGTPNWVDLQTTDQSAA

SEQ ID NO: 33 - DNA, 75 bp, Mycobacterium tuberculosis
gatcagtccgccgccaaaaagttctacacatcgttgttcggctggggttacgacgacaacccggtccccggaggc

SEQ ID NO: 34 - protein, 25 aa, Mycobacterium tuberculosis
DQSAAKKFYTSLFGWGYDDNPVPGG

SEQ ID NO: 35 - DNA, 75 bp, Mycobacterium tuberculosis
ccggtccccggaggcggtggggtctattccatggccacgctgaacggcgaagccgtggccgccatcgcaccgatg

SEQ ID NO: 36 - protein, 25 aa, Mycobacterium tuberculosis
PVPGGGGVYSMATLNGEAVAAIAPM

SEQ ID NO: 37 - DNA, 75 bp, Mycobacterium tuberculosis
gccatcgcaccgatgcccccgggtgcaccggaggggatgccgccgatctggaacacctatatcgcggtggacgac

SEQ ID NO: 38 - protein, 25 aa, Mycobacterium tuberculosis
AIAPMPPGAPEGMPPIWNTYIAVDD

SEQ ID NO: 39 - DNA, 75 bp, Mycobacterium tuberculosis
atcgcggtggacgacgtcgatgcggtggtggacaaggtggtgcccgggggcgggcaggtgatgatgccggccttc

SEQ ID NO: 40 - protein, 25 aa, Mycobacterium tuberculosis
IAVDDVDAVVDKVVPGGGQVMMPAF

SEQ ID NO: 41 - DNA, 75 bp, Mycobacterium tuberculosis
atgatgccggccttcgacatcggcgatgccggccggatgtcgttcatcaccgatccgaccggcgctgccgtgggc

SEQ ID NO: 42 - protein, 25 aa, Mycobacterium tuberculosis
MMPAFDIGDAGRMSFITDPTGAAVG

SEQ ID NO: 43 - DNA, 75 bp, Mycobacterium tuberculosis
ggcgctgccgtgggcctatggcaggccaatcggcacatcggagcgacgttggtcaacgagacgggcacgctcatc

SEQ ID NO: 44 - protein, 25 aa, Mycobacterium tuberculosis
GAAVGLWQANRHIGATLVNETGTLI

SEQ ID NO: 45 - DNA, 75 bp, Mycobacterium tuberculosis
acgggcacgctcatctggaacgaactgctcacggacaagccggatttggcgctagcgttctacgaggctgtggtt

SEQ ID NO: 46 - protein, 25 aa, Mycobacterium tuberculosis
TGTLIWNELLTDKPDLALAFYEAVV

SEQ ID NO: 47 - DNA, 75 bp, Mycobacterium tuberculosis
cgggtgctcaaggccggcgacgcggaagtcggcggctgtatggaaccgccgatgcccggcgtgccgaatcattgg

SEQ ID NO: 48 - protein, 25 aa, Mycobacterium tuberculosis
RVLKAGDAEVGGCMEPPMPGVPNHW

SEQ ID NO: 49 - DNA, 75 bp, Mycobacterium tuberculosis
gtgccgaatcattggcacgtctactttgcggtggatgacgccgacgccacggcggccaaagccgccgcagcgggc

SEQ ID NO: 50 - protein, 25 aa, Mycobacterium tuberculosis
VPNHWHVYFAVDDADATAAKAAAAG

SEQ ID NO: 51 - DNA, 75 bp, Mycobacterium tuberculosis
gccgccgcagcgggcggccaggtcattgcggaaccggctgacattccgtcggtgggccggttcgccgtgttgtcc

SEQ ID NO: 52 - protein, 25 aa, Mycobacterium tuberculosis
AAAAGGQVIAEPADIPSVGRFAVLS

SEQ ID NO: 53 - DNA, 63 bp, Mycobacterium tuberculosis
ttcgccgtgttgtccgatccgcagggcgcgatcttcagtgtgttgaagcccgcaccgcagcaa

SEQ ID NO: 54 - protein, 21 aa, Mycobacterium tuberculosis
FAVLSDPQGAIFSVLKPAPQQ

SEQ ID NO: 55 - protein, 35 aa, Artificial Sequence
  Description of Artificial Sequence  Primer
XGAAVGLWQANRHIGATLVNETGTLIWNELLTDKX

SEQ ID NO: 56 - protein, 34 aa, Artificial Sequence
  Description of Artificial Sequence  Primer
XCEPADIPSVGRFAVLSDPQGAIFSVLKPAPQQX

SEQ ID NO: 57 - DNA, 25 bp, Artificial Sequence
  Description of Artificial Sequence  Primer
atagaattcgatgcccaagagaagc

SEQ ID NO: 58 - DNA, 24 bp, Artificial Sequence
  Description of Artificial Sequence  Primer
attaagcttggcatcgccgatgtc

SEQ ID NO: 59 - DNA, 25 bp, Artificial Sequence
  Description of Artificial Sequence  Primer
attaggatccggccggatgtcgttc

SEQ ID NO: 60 - DNA, 23 bp, Artificial Sequence
  Description of Artificial Sequence  Primer
attaagcttttgctgcggtgcgg

SEQ ID NO: 61 - DNA, 26 bp, Artificial Sequence
  Description of Artificial Sequence  Primer
ataggatccggcggtggtggacaagg

SEQ ID NO: 62 - DNA, 26 bp, Artificial Sequence
  Description of Artificial Sequence  Primer
attaagcttagctatctccatgctcg

(19) United States
(12) Patent Application Publication
(10) Pub. No.: US 2005/0267025 A1
(43) Pub. Date: Dec. 1, 2005

(54) Compositions and methods for treatment of infectious and inflammatory diseases

(76) Inventor: John L. Ho, New York, NY (US)

Correspondence Address: Michael L. Goldman, Esq.;Nixon Peabody LLP, Clinton Square, P.O. Box 31051, Rochester, NY, 14603-1051, US

(21) Appl. No.: 11/083,624

(22) Filed: Mar. 18, 2005

Related U.S. Application Data
(63) Continuation-in-part of application No. 10/357,043, filed on Jan. 31, 2003.
(60) Provisional application No. 60/353,985, filed on Feb. 1, 2002.

(51) Int. Cl. A61K 38/17

(52) U.S. Cl. 514/12

(57) ABSTRACT

The present invention relates to a vaccine for preventing Mycobacterium tuberculosis infection that contains a polypeptide isolated from Mycobacterium tuberculosis that suppresses the immune response and induces IL-10 in a subject infected with Mycobacterium tuberculosis, and to methods of treating an inflammatory condition in a mammal, vaccinating a mammal against infection by Mycobacterium tuberculosis, and treating a mammal for infection by Mycobacterium tuberculosis, which involve providing a nucleic acid molecule encoding the polypeptide isolated from Mycobacterium tuberculosis that suppresses the immune response and induces IL-10 in a subject infected with Mycobacterium tuberculosis, or the polypeptide itself, and administering the nucleic acid molecule or its encoded polypeptide to a mammal.

This application is a continuation-in-part of U.S. patent application Ser. No. 10/357,043, filed Jan. 31, 2003, which claims benefit of U.S. Provisional Patent Application Ser. No. 60/353,985, filed Feb. 1, 2002, which is hereby incorporated by reference in its entirety.

The subject matter of this application was made with support from the United States Government under the National Institutes of Health Grant Nos. A139606, HL61960, and TW0018. The U.S. Government may have certain rights.

### FIELD OF THE INVENTION

The present invention relates to compositions and methods for the prevention and treatment of Mycobacterium tuberculosis infection and inflammatory disease conditions in mammals.

### BACKGROUND OF THE INVENTION

Control of Mycobacterium tuberculosis (Mtb), the etiologic agent of tuberculosis (Tb), is immune cell mediated as shown by humans without a functioning interferon gamma receptor (IFN-γR) or interleukin-12 receptor (IL-12R) manifesting with disseminated mycobacteria disease (Dorman et al., “Interferon-Gamma and Interleukin-12 Pathway Defects and Human Disease,” Cytokine Growth Factor Rev. 11(4):321-33 (2000); Jouanguy et al., “IL-12 and IFN-Gamma in Host Defense Against Mycobacteria and Salmonella in Mice and Men,” Curr. Opin. Immunol. 11(3):346-51 (1999); Altare et al., “Inherited Interleukin 12 Deficiency in a Child with Bacille Calmette-Guerin and Salmonella Enteritidis Disseminated Infection,” J. Clin. Invest. 102(12):2035-40 (1998); Sakai et al., “Missense Mutation of the Interleukin-12 Receptor Beta 1 Chain-Encoding Gene is Associated with Impaired Immunity Against Mycobacterium avium Complex Infection,” Blood 97(9):2688-94 (2001)). Moreover, immunosuppression by drugs, cancer, HIV-1 or immune senescence is associated with reactivation Tb, highlighting the fact that Mtb avoids immune elimination to establish life-long infection (Rook et al., “Advances in the Immunopathogenesis of Pulmonary Tuberculosis,” Curr. Opin. Pulm. Med. 7(3):116-23 (2001); Flynn et al., “Immunology of Tuberculosis,” Annu. Rev. Immunol. 19:93-129 (2001); Ho et al., “Defenses of the Lung Against Tuberculosis,” in The Lung: Scientific Foundations, Crystal et al., eds., 2nd Edition, Chapter 183, pp. 2381-94 (1997); Vanham et al., “Examining a Paradox in the Pathogenesis of Human Pulmonary Tuberculosis: Immune Activation and Suppression/Anergy,” Tuber. Lung Dis. 78(3-4):145-58 (1997); Ellner, “Regulation of the Human Immune Response During Tuberculosis,” J. Lab. Clin. Med. 130(5):469-75 (1997)). This accounts for one in three persons worldwide having latent Mtb infection and a 5-10% lifetime risk of progression to active disease, translating to ˜8 million annual active Tb cases and ˜3 million annual deaths (Bishai, “The Mycobacterium tuberculosis Genomic Sequence: Anatomy of a Master Adaptor,” Trends Microbiol. 6(12):464-5 (1998)). Genes present in Mtb but absent in non-pathogenic mycobacteria are proposed as virulence factors. However, which Mtb specific genes mediate rapid progression to disease or transit to latent infection, and how these genes function, remain poorly defined.

There are several examples whereby specific gene products of microbes modulate the host immune response to effect microbial survival (Orth et al., “Disruption of Signaling by Yersinia Effector YopJ, a Ubiquitin-Like Protein Protease,” Science 290(5496):1594-7 (2000); Boland et al., “Role of YopP in Suppression of Tumor Necrosis Factor Alpha Release by Macrophages During Yersinia Infection,” Infect. Immun. 66(5):1878-84 (1998); Cornelis et al., “Yersinia Lead SUMO Attack,” Nat. Med. 7:21-23 (2001); Trufariello et al., “Adenovirus E3 14.7-kDa Protein, an Antagonist of Tumor Necrosis Factor Cytolysis, Increases the Virulence of Vaccinia Virus in Severe Combined Immunodeficient Mice,” Proc. Natl. Acad. Sci. USA 91:10987-91 (1994); Trufariello et al., “Adenovirus E3 14.7-kDa Protein, an Antagonist of Tumor Necrosis Factor Cytolysis, Increases the Virulence of Vaccinia Virus in a Murine Pneumonia Model,” J. Virol. 68:453-62 (1994); Nigou et al., “Mannosylated Lipoarabinomannans Inhibit IL-12 Production by Human Dendritic Cells: Evidence for a Negative Signal Delivered through the Mannose Receptor,” J. Immunol 166(12):7477-85 (2001); Stockl et al., “Human Major Group Rhinoviruses Down-Modulate the Accessory Function of Monocytes by Inducing IL-10,” J. Clin. Invest. 104(7):957-65 (1999); Fleming et al., “A Homolog of Interleukin-10 is Encoded by the Poxvirus Orf Virus,” J. Virol. 71(6):4857-61 (1997); Vockerodt et al., “The Epstein-Barr Virus Latent Membrane Protein 1 Induces Interleukin-10 in Burkitt's Lymphoma Cells but not in Hodgkin's Cells Involving the p38/SAPK2 Pathway,” Virology 280(2):183-98 (2001); Henke et al., “Viral IL-10 Gene Transfer Decreases Inflammation and Cell Adhesion Molecule Expression in a Rat Model of Venous Thrombosis,” J. Immunol. 164(4):2131-41 (2000); Suzuki et al., “Viral Interleukin 10 (IL-10), the Human Herpes Virus 4 Cellular IL-10 Homologue, Induces Local Anergy to Allogeneic and Syngeneic Tumors,” J. Exp. Med. 182(2):477-86 (1995); Wynn et al., “Analysis of Granuloma Formation (by Schistomsoma eggs) in Double Cytokine-Deficient Mice Reveals a Central Role for IL-10 in Polarizing Both T Helper Cell 1- and T Helper Cell 2-Type Cytokine Responses In vivo,” J. Immunol. 159(10):5014-23 (1997); Barcova et al., “gp41 Envelope Protein of Human Immunodeficiency Virus Induces Interleukin (IL)-10 in Monocytes, but not in B, T, or NK Cells, Leading to Reduced IL-2 and Interferon-Gamma Production,” J. Infect. Dis. 177(4):905-13 (1998); Taoufik et al., “Human Immunodeficiency Virus gp120 Inhibits Interleukin-12 Secretion by Human Monocytes: an Indirect Interleukin-10-Mediated Effect,” Blood 89(8):2842-8 (1997); Koutsonikolis et al., “HIV-1 Recombinant gp41 Induces IL-10 Expression and Production in Peripheral Blood Monocytes but not in T-Lymphocytes,” Immunol. Lett. 55(2):109-13 (1997); Schols et al., “Human Immunodeficiency Virus Type 1 gp120 Induces Anergy in Human Peripheral Blood Lymphocytes by Inducing Interleukin-10 Production,” J. Virol. 70(8):4953-60 (1996)). Specifically, Epstein-Barr virus (EBV) encodes a human IL-10 homolog as well as the EBV latent protein-1 that induces IL-10 (Vockerodt et al., “The Epstein-Barr Virus Latent Membrane Protein 1 Induces Interleukin-10 in Burkitt's Lymnphoma Cells but not in Hodgkin's Cells Involving the p38/SAPK2 Pathway,” Virology 280(2):183-98 (2001); Henke et al., “Viral IL-10 Gene Transfer Decreases Inflammation and Cell Adhesion Molecule Expression in a Rat Model of Venous Thrombosis,” J. Immunol. 164(4):2131-41 (2000); Suzuki et al., “Viral Interleukin 10 (IL-10), the Human Herpes Virus 4 Cellular IL-10 Homologue, Induces Local Anergy to Allogeneic and Syngeneic Tumors,” J. Exp. Med. 182(2):477-86 (1995)). Both of these EBV factors are thought to facilitate viral survival and pathogenesis through IL-10's immune suppressive activity (Vockerodt et al., “The Epstein-Barr Virus Latent Membrane Protein 1 Induces Interleukin-10 in Burkitt's Lymphoma Cells but not in Hodgkin's Cells Involving the p38/SAPK2 Pathway,” Virology 280(2): 183-98 (2001); Henke et al., “Viral IL-10 Gene Transfer Decreases Inflammation and Cell Adhesion Molecule Expression in a Rat Model of Venous Thrombosis,” J. Immunol. 164(4):2131-41 (2000); Suzuki et al., “Viral Interleukin 10 (IL-10), the Human Herpes Virus 4 Cellular IL-10 Homologue, Induces Local Anergy to Allogeneic and Syngeneic Tumors,” J. Exp. Med. 182(2):477-86 (1995)). IL-10 is a potent inhibitor of inflammatory response to pathogens, suppressing the production of cytokines such as tumor necrosis factor alpha (TNF-α), IL-12, IFN-γ and expression of macrophage NOS2 as well as costimulatory molecules such as CD40, CD80, and CD86, immune factors involved in control of Mtb infection (Brossart et al., “Tumor Necrosis Factor −α and CD40 Ligand Antagonize the Inhibitory Effects of Interleukin 10 and T-Cell Stimulatory Capacity of Dendritic Cess 1.,” Can. Res. 60:4485-92 (2000); Gao et al., “CD40-Deficient Dendritic Cells Producing Interleukin-10, but not Interleukin-12, Induce T-cell Hyporesponsiveness In vitro and Prevent Acute Allograft Rejection,” Immunology 98(2):159-70 (1999); Villegas et al., “Blockade of Costimulation Prevents Infection-Induced Immunopathology in IL-10-Deficient Mice,” Infect. Immun. 68:2837-44 (2000); Van Gool et al., “Blocking CD40-CD154 and CD80/CD86 -CD28 Interactions During Primary Allogeneic Stimulation Results in T Cell Anergy and High IL-10 Production,” Eur. J Immunol. 29(8):2367-75 (1999); Akdis et al., “Mechanisms of Interleukin-10-Mediated Immune Suppression,” Immunology 103(2):131-6 (2001)). In addition, there is growing evidence that IL-10 is involved in Tb. Specifically, IL-10 plays a critical role in murine model of M. bovis Bacillus Calmitte-Guerin (BCG) infection, because IL-10 over-expression enhanced bacilli growth while IL-10 depletion by gene knock-out (KO) increased anti-mycobacterial immunity and lowered BCG load (Murray et al., “Increased Antimycobacterial Immunity in Interleukin-10-Deficient Mice,” Infect. Immun. 67(6):3087-95 (1999); Murray et al., “T Cell-Derived IL-10 Antagonizes Macrophage Function in Mycobacterial Infection,” J. Immunol. 158(l):315-21 (1997); Jacobs et al., “Increased Resistance to Mycobacterial Infection in the Absence of Interleukin-10,” Immunology 100(4):494-501(2000)). Clinical data also lend support for IL-10 in Tb pathogenesis, because neutralization of IL-10 from peripheral blood cells from active Tb patients enhanced Mtb specific T cell proliferation and IFN-γ production and increased monocyte production of IL-12 and CTLA-4 expression (Samten et al., “Depressed CD40 Ligand Expression Contributes to Reduced Gamma Interferon Production in Human Tuberculosis,” Infect. Immun. 68(5):3002-6 (2000); Gong et al., “Interleukin-10 Downregulates Mycobacterium tuberculosis-Induced Th1 Responses and CTLA-4 Expression,” Infect. Immun. 64(3):913-8 (1996)), IL-10 mediates the anergy seen in some patients with active Tb (Baliko et al., “Th2 Biased Immune Response in Cases with Active Mycobacterium tuberculosis Infection and Tuberculin Anergy,” FEMS Immunol. Med. Microbiol. 22(3): 199-204 (1998); Boussiotis et al., “IL-10-Producing T Cells Suppress Immune Responses in Anergic Tuberculosis Patients,” J. Clin. Invest. 105(9): 1317-25 (2000)), predominant T cell clones obtained from the lungs of active Tb cases secrete both IL-10 and IFN-γ (Rook et al., “Advances in the Immunopathogenesis of Pulmonary Tuberculosis,” Curr. Opin. Pulm. Med. 7(3):116-23 (2001); Ho et al., “Defenses of the Lung Against Tuberculosis,” in The Lung: Scientific Foundations, Crystal et al., eds., 2nd Edition, Chapter 183, pp. 2381-94 (1997); McAdam et al., “Polarization of PPD-Specific T-Cell Response of Patients with Tuberculosis from Th0 to Th1 Profile After Successful Antimycobacterial Therapy or In vitro Conditioning with Interferon-Alpha or Interleukin-12,” Am. J. Respir. Cell Mol. Biol. 24(2):187-94 (2001)), and IL-10 production is triggered by Mtb infection (Gong et al., “Interleukin-10 Downregulates Mycobacterium tuberculosis-Induced Th1 Responses and CTLA-4 Expression,” Infect. Immun. 64(3):913-8 (1996); Almeida et al., “Induction of In vitro Human Macrophage Primed-Lymphocytes,” J. Immunol. 160:4490-9 (1998); Fulton et al., “Regulation of Interleukin-12 by Interleukin-10, Transforming Growth Factor-Beta, Tumor Necrosis Factor-Alpha, and Interferon-Gamma in Human Monocytes Infected with Mycobacterium tuberculosis H37Ra,” J. Infect. Dis. 178(4):1105-14 (1998); Giacomini et al., “Infection of Human Macrophages and Dendritic Cells with Mycobacterium tuberculosis Induces a Differential Cytokine Gene Expression That Modulates T Cell Response,” J. Immunol. 166(12):7033-41 (2001)).

Macrophages are the preferred cell for intracellular survival of Mtb. It is also recognized that the macrophage and Mtb interaction may be critical to the outcome of infection by Mtb. This is underscored by the finding that depletion of alveolar macrophages in mice exerted protective effects for pulmonary Tb (Leemans et al., “Depletion of Alveolar Macrophages Exerts Protective Effects in Pulmonary Tuberculosis in Mice,” J. Immunol. 166(7):4604-11 (2001)) and patients with silicosis (where lung macrophages are paralyzed by the inhaled silicate) have an increased risk for active Tb (Davies, “Silicosis and Tuberculosis Among South African Goldminers—An Overview of Recent Studies and Current Issues,” S. Afr. Med. J. 91(7):562-6 (2001) Review)). In addition, murine models of Tb have shown that susceptible mice (Balb/C or I/St), in contrast to resistant mice (C56B16 or A/Sn), produced higher amounts of IL-10, lower amounts of IFN-γ and IL-12, and their macrophages expressed lower NOS2, thereby contributing to the severity of disease (Yoshida et al., “Dissection of Strain Difference in Acquired Protective Immunity Against Mycobacterium bovis Calmette-Guerin Bacillus (BCG). Macrophages Regulate the Susceptibility Through Cytokine Network and the Induction of Nitric Oxide Synthase,” J. Immunol. 155(4):2057-66 (1995)). Several groups have reported that in vitro Mtb infection of human monocyte/macrophages is associated with high IL-10 production. In addition, in a human cell culture model of immune control of MtbH37Ra infection, low IL-10 production was associated with reduction in bacilli load while high IL-10 was associated with uncontrolled growth of Mtb.

What is needed now is clear evidence that the Rv0577 gene of Mtb is an immunomodulatory factor in Mtb infection, and methods which utilize this gene, and its protein product, for the detection, prevention, and treatment of Mycobacterium tuberculosis infection and other inflammatory disease conditions.

The present invention is directed to overcoming these and other deficiencies in the art.

### SUMMARY OF THE INVENTION

The present invention relates to a nucleic acid construct having a nucleic acid molecule that encodes a factor suppressing an immune response to Mycobacterium tuberculosis in a host subject, where the nucleic acid molecule either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of at least 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2; and has an operably linked DNA promoter and an operably linked 3′ regulatory region.

The present invention also relates to an isolated antibody, or binding portion thereof, against a protein or polypeptide having an amino acid corresponding to SEQ ID NO: 2.

Another aspect of the present invention is a method for detection of Mycobacterium tuberculosis specific antibodies in a sample of tissue or body fluids. This method involves providing an isolated protein or polypeptide having an amino acid corresponding to SEQ ID NO: 2 as an antigen; contacting the sample with the antigen under conditions effective to allow formation of a complex of the antigen bound to antibodies which recognize the antigen; and detecting if any of the complex is present, thereby indicating a presence of Mycobacterium tuberculosis the sample.

The present invention also relates to another method for detection of Mycobacterium tuberculosis in a sample of tissue or body fluids. This method involves providing an antibody or binding portion thereof against the protein or polypeptide of the present invention having an amino acid corresponding to SEQ ID NO: 2, contacting the sample with the antibody or binding portion thereof under conditions effective to allow formation of a complex of the antibody or binding portion thereof and an antigen recognized by the antibody or binding portion thereof, and detecting if any of the complex is present, thereby indicating a presence of Mycobacterium tuberculosis in the sample.

The present invention also relates to a third method for detection of Mycobacterium tuberculosis in a sample of tissue or body fluids. This method involves providing a nucleic acid molecule as a probe in a nucleic acid hybridization assay; contacting the sample with the probe under conditions effective to permit formation of a complex of the probe and nucleic acid which hybridizes to the probe; and detecting formation of the complex in the sample, thereby indicating a presence of Mycobacterium tuberculosis in the sample. The nucleic acid molecule either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of at least 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2.

The present invention also relates to a fourth method of detection of Mycobacterium tuberculosis in a sample of tissue or body fluids. This method involves providing a nucleic acid molecule as a probe or primer in a gene amplification detection procedure, contacting the sample with the probe or primer under conditions effective to amplify probe or primer-specific nucleic acid molecules; and detecting any amplified probe or primer-specific molecules, thereby indicating a presence of Mycobacterium tuberculosis in the sample. The nucleic acid molecule either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of at least 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2.

The present invention also relates to a method of vaccinating a mammal against infection by Mycobacterium tuberculosis. This method involves administering an effective amount of an isolated protein or polypeptide having an amino acid sequence corresponding to SEQ ID NO: 2 to the mammal.

Another aspect of the present invention is a vaccine for preventing infection and disease of mammals by Mycobacterium tuberculosis. This vaccine includes an isolated protein or polypeptide having an amino acid sequence corresponding to SEQ ID NO: 2; and a pharmaceutically-acceptable carrier.

Another aspect of the present invention is a method of vaccinating mammals against infection by Mycobacterium tuberculosis. This involves administering to mammals an effective amount of the vaccine of the present invention that includes an isolated protein or polypeptide having an amino acid sequence corresponding to SEQ ID NO: 2 and a pharmaceutically-acceptable carrier.

Another aspect of the present invention is a method of treating mammals infected with Mycobacterium tuberculosis. This method involves administering an effective amount of the isolated antibody, or binding portion thereof, against a protein or polypeptide having an amino acid corresponding to SEQ ID NO: 2, to mammals infected with Mycobacterium tuberculosis.

Another aspect of the present invention is a composition for passively immunizing mammals infected with Mycobacterium tuberculosis. This composition includes an isolated antibody, or binding portion thereof, against a protein or polypeptide having an amino acid corresponding to SEQ ID NO: 2, and a pharmaceutically-acceptable carrier.

Another aspect of the present invention is a method for passively immunizing mammals infected with Mycobacterium tuberculosis. This method involves administering an effective amount of the composition of the present invention having an isolated antibody, or binding portion thereof, against a protein or polypeptide having an amino acid corresponding to SEQ ID NO: 2, and a pharmaceutically-acceptable carrier.

Another aspect of the present invention relates to a method of enhancing vaccination against Mycobacterium tuberculosis using a composition comprising a microorganism capable of producing an antigenic response against Mycobacterium tuberculosis when introduced into a host subject. This method involves suppressing in the microorganism the expression of a nucleic acid molecule that either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of at least 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2.

The present invention also relates to a composition for actively immunizing mammals against Mycobacterium tuberculosis. This composition has a microorganism capable of producing an antigenic response against Mycobacterium tuberculosis when introduced into a host subject, where the microorganism has been modified to be incapable of producing a nucleic acid molecule encoding a factor suppressing an immune response to Mycobacterium tuberculosis in a host, and a pharmaceutically-acceptable carrier.

Another aspect of the present invention relates to a method of vaccinating a mammal against infection by Mycobacterium tuberculosis. This method involves administering an effective amount of a composition having a microorganism capable of producing an antigenic response against Mycobacterium tuberculosis, where the microorganism has been modified to be incapable of producing a nucleic acid molecule encoding a factor suppressing an immune response to Mycobacterium tuberculosis in a host, and a pharmaceutically-acceptable carrier.

Another aspect of the present invention is a method of treating inflammatory disease in a mammal. This method involves providing a nucleic acid construct having a nucleic acid molecule that encodes a factor suppressing an immune response to Mycobacterium, where the nucleic acid molecule either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of at least 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2; and operably linked 5′ and 3′ regulatory elements. The nucleic acid construct is administered to a mammal under conditions effective to treat an inflammatory disease.

The present invention also relates to another method of treating inflammatory disease in a mammal. This method involves providing a protein or polypeptide that suppresses an immune response to Mycobacterium tuberculosis, where the protein or polypeptide has an amino acid sequence of SEQ ID NO: 2; and administering the protein or polypeptide to a mammal under conditions effective to treat an inflammatory disease.

The present invention also relates a method of treating an inflammatory condition in a mammal that involves providing a polypeptide having either: 1) the amino acid sequence of SEQ ID NO: 34; 2) the amino acid sequence of SEQ ID NO: 40; or 3) the amino acid sequence of SEQ ID NO: 44; and administering the polypeptide to a mammal under conditions effective to treat the inflammatory condition.

The present invention also relates to a method of treating an inflammatory condition in a mammal that involves providing a nucleic acid construct including a nucleic acid having either: 1) the nucleotide sequence of SEQ ID NO: 33; 2) the nucleotide sequence of SEQ ID NO: 39; 3) the nucleotide sequence of SEQ ID NO: 43; 4) a nucleic acid molecule encoding a polypeptide having the amino acid sequence of SEQ ID NO: 34; 5) a nucleic acid molecule encoding a polypeptide having the amino acid sequence of SEQ ID NO: 40; or 6) a nucleic acid molecule encoding a polypeptide having the amino acid sequence of SEQ ID NO: 44, and an operably linked DNA promoter and 3′ regulatory region. The method also involves administering the nucleic acid construct to a mammal under conditions effective to treat the inflammatory condition.

The present invention also provides a method of vaccinating a mammal against infection by Mycobacterium tuberculosis. This method involves administering to the mammal an effective amount of an isolated polypeptide, where the polypeptide includes either: 1) the amino acid sequence of SEQ ID NO: 34; 2) the amino acid sequence of SEQ ID NO: 40; or 3) the amino acid sequence of SEQ ID NO: 44.

The present invention also relates to a vaccine for preventing Mycobacterium tuberculosis infection and disease in mammals. The vaccine includes an isolated polypeptide having either: 1) the amino acid sequence of SEQ ID NO: 34; 2) the amino acid sequence of SEQ ID NO: 40; or 3) the amino acid sequence of SEQ ID NO: 44.

The present invention also relates to a method of vaccinating mammals against infection by Mycobacterium tuberculosis. This method includes administering an effective amount of the vaccine including an isolated polypeptide having either: 1) the amino acid sequence of SEQ ID NO: 34; 2) the amino acid sequence of SEQ ID NO: 40; or 3) the amino acid sequence of SEQ ID NO: 44, to mammals.

Another aspect of the present invention is a method of treating mammals infected with Mycobacterium tuberculosis. This method involves administering an effective amount of the antibody or binding portion thereof raised against a polypeptide having either: 1) the amino acid sequence of SEQ ID NO: 34; 2) the amino acid sequence of SEQ ID NO: 40; or 3) the amino acid sequence of SEQ ID NO: 44 to mammals infected with Mycobacterium tuberculosis, thereby treating the infection.

The present invention also relates to a method of enhancing vaccination against Mycobacterium tuberculosis. This method involves suppressing in the microorganism the expression of a nucleic acid molecule having either: 1) the nucleotide sequence of SEQ ID NO: 33; 2) the nucleotide sequence of SEQ ID NO: 39; 3) the nucleotide sequence of SEQ ID NO: 43; 4) a nucleic acid molecule encoding a polypeptide having the amino acid sequence of SEQ ID NO: 34; 5) a nucleic acid molecule encoding a polypeptide having the amino acid sequence of SEQ ID NO: 40; or 6) a nucleic acid molecule encoding a polypeptide having the amino acid sequence of SEQ ID NO: 44, and administering the suppressed microorganism to a subject under conditions effective to enhance vaccination against Mycobacterium tuberculosis.

### BRIEF DESCRIPTION OF THE DRAWINGS

FIG. 1 is a graph comparing IL-10 induction by M. tuberculosis (Mtb) and M. smegmatis (Ms). Induction of IL-10 by mycobacteria was performed using human peripheral blood monocytes from a leukocyte rich blood bank preparation purified by negative selection (up to 90% pure by CD14 expression on FACS analysis) and cultured in X-Vivo-20 medium (BioWhittiker, an artificial medium without protein or detectable endotoxin). The results shown are the mean±SD of M. smegmatis (Ms, ATCC No. 23038, lots 961, 972, or mc² 155), Mtb H37Rv (MtbRv, ATCC 27294, lots 013, 082) or MtbH37Ra (MtbRa, ATCC 25177, lot 082), at 0.5 colony forming units (cfu) stimulation per monocyte at 10⁶ monocytes per well (in duplicate) from 5 to 8 separate donors, P<0.03 for MtbRv or MtbRa compared to Ms; P>0.05 for Ms versus medium, Students' paired t-test. LPS (E. coli lipopolysaccharide, 1 82 g/ml).

FIGS. 2A-C are graphs showing IL-10 inducing activity by Mtb H37Rv preparations. FIG. 2A shows IL-10 production in cell-free supernatants from cultures of blood bank donor monocytes (1×10⁶/well, isolated by self-aggregation method) at 48 h after stimulation (most in triplicates) with Mtb H37Rv (0.5 cfu per monocyte) or with Mtb H37Rv components, cell wall (1 μg/ml), cytosol (1 μg/ml), membrane (1 μg/ml), or culture filtrate (CFP, 1 μg/ml) and purified protein derivative (PPD, 1 μg/ml) of Mtb. FIG. 2B shows the effect of varying doses of MtbH37Rv CFP on IL-10 production. FIG. 2C shows enrichment of IL-10 activity by anion exchange chromatography. Monocytes were stimulated with 0.2 μg/ml of each fraction (fx) of MtbH37Rv CFP produced by anion exchange (QAE) chromatography. CFP fractionated by anion exchange (QAE) chromatography. The results are the mean±SD of indicated number (n) of donors.

FIGS. 3A-C are graphs showing the induction of cytokines by MtbH37Rv or preparations of Mtb. Cell-free supernatants obtained from blood bank donor monocytes (1×10⁶/well, self-aggregation method) at 48 h after stimulation (in triplicate) with MtbH37Rv mannose capped lipoarabinomannan (manLAM, 5 μg/ml), MtbH37Rv CFP (0.5 to 1 μg/ml) or MtbH37Rv CFP fx9 by anion exchange chromatography (0.2 μg/ml), or 0.5 cfu MtbH37Ra per monocytes. FIG. 3A shows IL-10 assay results. FIG. 3B shows TNF-α assay results. FIG. 3C shows assay results using IL-1β antibodies. All results are the mean±SD of 8 to 14 donors tested with each Mtb reagent.

FIGS. 4A-E show the creation and growth of 577 null Mtb and a complemented 577 null mutant Mtb. FIG. 4A is a Southern blot of genomic DNA digested by PVUII, separated by electrophoresis, transferred to membrane and analyzed by Southern blot performed using a digoxitonin-labeled Rv0577 probe obtained by PCR with detection by chemiluminescence. FIG. 4B is a PCR amplification analysis using gene amplification primer pairs previously reported to detect all mycobacteria (16S rRNA), only Mtb complex subspecies (MPB70), only M. smegmatis (Ms0911), Rv0577 (cfp32), or the insertion of Rv0577 into the multiple cloning site of pMSG (MCS pMSG). FIG. 4C is a Western blot analysis of the parental MtbH37Rv, Rv0577 null mutant, and Rv0577 complemented null mutant. The Rv0577 null mutant was transformed with pMSG.577 plasmid in which Rv0577 expression is under the regulation of the constitutive Mtb glutamine synthase promoter. Cell lysates (1 μg) were separated by electrophoresis on Tris-Bis acrylamide, transferred to nitrocellulose, and recombinant Rv0577 or unknown sample were probed with rabbit polyclonal anti-rRv0577 antisera (3^{rd} bleed) and developed with anti-rabbit Ab linked to horseradish peroxidase chemiluminescent assay. FIG. 4D is a graph showing the growth kinetics of parental, 577 null mutant, and complementated 577 null mutant Mtb, quantified by OD580 nm of cultures inoculated into 7H9 broth supplemented with ADC (plus hygromycin B 50 μg/ml for 577 null mutant or plus kanamycin 25 μg/ml for complementated 577 null mutant). FIG. 4E shows the results of the broth cultures from FIG. 4D after being plated on 7H11 agar supplemented with OADC and antibiotics. Illustrated are the mean±SD of three independent experiments. FIGS. 4F-H show the colony morphology of parental, 577 null mutant, and complementated 577 null mutant Mtb, respectively, grown on 7H11 agar.

FIGS. 5A-B show the characterization of Rv0577. FIG. 5A shows IL-10 production resulting from a challenge by 577 null mutant compared with parental was statistically significant (n=7 separate donors, mean±SE; P≦0.01, Students' paired t-test). FIG. 5B shows TNF-α production assayed using the same culture supernatants; P>0.05, mean±SD. Induction of IL-10 by mycobacteria or LPS was performed using human peripheral blood monocytes (10⁶ per well in 1 ml) purified by negative selection and cultured in X-Vivo-20 medium. Parental, Rv0577 null mutant, Rv0577 complemented (pMSG.577), null mutant or laboratory assay standard Mtb H37Rv at 0.1 or 0.5 cfu per monocyte were compared with medium control or LPS (100 ng/ml) stimulation of monocyte production of IL-10 or TNF-α at 48 h.

FIGS. 6A-E show the results of over-expression of Rv0577 in M. smegmatis and the effect of Rv0577 overexpression on IL-10 and TNF-α production. FIG. 6A shows gene amplification by PCR analysis of parental M. smegmatis (Ms) and M. smegmatis transformants possessing pMS3.577 and pMS3 plasmids. The pMS3 plasmid contains the hygromycin resistance gene under the control of the constitutive M. smegmatis heat shock protein promoter. Gene amplification utilized primer pairs as described for FIG. 4B, supra, and primer pairs for the backbone of pMS3 in order to visualize a backbone DNA fragment or the backbone plus a DNA insert. FIG. 6B is a Western blot analysis of cell lysates (1 μg amounts) of the parental M. smegmatis and M. smegmatis transformants possessing pMS3.577 and pMS3 plasmids, studied for expression of Rv0577 protein as detailed in FIG. 4 legend, supra. FIG. 6C is a graph showing IL-10 production of M. smegmatis (Ms) infected human monocytes in comparison to MtbRvH37 infected and LPS-treated monocytes. In FIGS. 6D-E, M. smegmatis (strain MC²155) transformed with pMS3 plasmid or pMS3.577 were grown in 7H11 medium containing hygromycin 10 μg/ml, and washed bacilli were used to generate whole cell lysate or to infected monocytes. Human monocytes freshly isolated by negative selection were infected with M. smegmatis transformants containing pMS3 or pMS3.577 plasmid at 0.04 to 0.05 cfu to monocyte ratio. Cell supernatant were assayed for IL-10, shown in FIG. 6D, or TNF-α production, shown in FIG. 6E (mean±SD; n=4, P≦0.01, using absolute IL-10 values between M. smegmatis transformants containing pMS3 or pMS3.577).

FIGS. 7A-C characterize CFP32 active peptides. FIG. 7A is the amino acid sequence of CFP32 (SEQ ID NO: 2) showing the amino acid sequence (SEQ ID NO: 44) of peptide 7 (pep7) underlined. FIG. 7B is graph showing IL-10 inducing activity of overlapping CFP32 peptides. FIG. 7C is a hydrophilicity plot of CFP32 and peptides: pep2 (* over bar),-pep5 († over bar) and -pep7 (‡ over bar).

FIG. 8 is western blot showing immunoreactivity of antisera raised against Rv0577 whole recombinant CFP32 protein or peptide domains of the CFP32 protein. Native Rv0577 from MtbH37 lysate and rRv0577 resolved on 15% SDS PAGE transferred to a nitrocellulose membrane were analyzed by WB using preimmune sera or immune sera as described in Example 9.

FIG. 9 is a diagram showing the Rv0577 gene and the location of active peptides 5 and 7, and subunits N(330), M(262), and C(542) cloned for CFP32 protein expression testing.

FIGS. 10A-C show the expression of recombinant Rv0577/CFP32 subunit proteins in transformed E. coli. Protein expression was monitored in Luria broth cultures under IPTG induction (+) or uninduced growth conditions (−). FIG. 10A shows IPTG induced expression of a 10 kD protein from pET23bN330 transformed E. coli FIG. 10B shows IPTG induced (+) expression of a 15 kD protein by E. coli transformed with pQE30C456. FIG. 10C shows IPTG induced (+) expression of an 8.7 kD protein from pET23bC262 transformed E. coli (FIG. 10C).

FIGS. 11A-B show western blot analyses of rCFP32 whole protein, and subunit proteins N330 and C456. Western blot was performed using the murine anti-rC456, shown in FIG. 11A, or anti-rN330 shown in FIG. 11B, Rv0577 subunit sera. Results show whole CFP32 protein was recognized by anti-N330 and anti-C456, each antibody recognized its appropriate antigen protein, but no cross-reactivity occurred between subunits.

FIGS. 12A-B show stimulation of IL-10 production in monocytes. FIG. 12A shows IL-10 production in freshly isolated monocytes stimulated at varying doses (in duplicate) of culture filtrate with culture filtrates (CF) from wildtype (WT) and 577Null mutant Mtb H37 Rv. FIG. 12B shows IL-10 production in monocytes treated with CFP alone or filtrate or retentate partitioned by 100 kDa membrane filtration of CFP plus rabbit polyclonal antisera raised against recombinant CFP32 (α-rCFP32) incubated for 1 hr and passed through a 100 KDa column to derive a filtrate and retentate. All results are the mean±SD for (n) number of donors performed in duplicate, P<0.01, WT versus 577Null CFP doses, and WT CFP versus retentate.

FIGS. 13A-C show heterologous trans-expression of CFP32 in yeast (Pischia pastoris) measured as ability to induce IL-production in monocytes. FIG. 13A is a western blot showing exogenous production of CFP32 by pPICZ.577-transformed yeast (α-rCFP32-E. coli expressed as primary anti-serum, 1 μg of Mtb H37Rv culture filtrate (CF), 10 ng of His-tag purified yeast rCFP32, and 50 ng of concentrated filtered yeast culture medium in which yeast transformed with the missense CFP32 expression vector were grown [BMMY]). Band sizes of markers (kDa) are indicated at left. FIG. 13B shows the ability of the histidine (His)-tagged purified yeast rCFP32 to induce IL-10 production (measured by ELISA) in monocytes in a dose-dependent manner. Filtrate and retentate were used to challenge monocytes and IL-10 levels at 48 h were measured by ELISA. All results are the mean±SD for (n) number of donors performed in duplicate.

FIG. 14 shows an assay demonstrating CFP32-mediated inhibition of antimicrobial activity by monocytes. Monocytes were previously treated with yeast rCFP32, culture filtrates proteins (CFP) from Mtb H37Rv wildtype (WT), 577Null mutant and 577complemented 577Null mutant (577Comp or Comp). Human (hu) rIL-10 and heat killed S. aureus cowan strain (SAC) served as positive controls. Illustrated are the mean and SD respectively for n=6, 5, and 12 paired values; P=* 0.005, **0.03, and †0.002, two-tailed t-test.

FIGS. 15A-B show the production of IL-6 induced in macrophage-like (glioma) cell lines treated with yeast-expressed rCFP32, LPS (100 ng/ml), peptidoglycan (15 μg/ml, PGN), and BMMY (medium from yeast transformed with plasmid having an out-of-frame CFP32 insert). FIG. 15A shows IL-6 production U373/CD14 cells. FIG. 15B shows IL-6 production in U373/CD14/TLR2 cells. Illustrated are the mean and SD of two separate experiments performed in duplicate.

### DETAILED DESCRIPTION OF THE INVENTION

The present invention relates to a nucleic acid construct having a nucleic acid molecule that encodes a factor suppressing an immune response to Mycobacterium tuberculosis in a host subject, where the nucleic acid molecule either: 1) has a nucleotide sequence corresponding to SEQ ID NO: 1 herein; 2) has a nucleotide sequence that hybridizes to the nucleic acid corresponding to SEQ ID NO: 1 under stringent conditions characterized by a hybridization buffer comprising 5×SSC at a temperature of at least 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2 herein, and has an operably linked DNA promoter and an operably linked 3′ regulatory region.

One isolated nucleotide sequence suitable as a nucleic acid molecule of the construct of the present invention has a nucleotide sequence of SEQ ID NO: 1, as follows:

atgcccaaga gaagcgaata caggcaaggc acgccgaact gggtcgacct tcagaccacc 60
gatcagtccg ccgccaaaaa gttctacaca tcgttgttcg gctggggtta cgacgacaac 120
ccggtccccg gaggcggtgg ggtctattcc atggccacgc tgaacggcga agccgtggcc 180
gccatcgcac cgatgccccc gggtgcaccg gaggggatgc cgccgatctg gaacacctat 240
atcgcggtgg acgacgtcga tgcggtggtg gacaaggtgg tgcccggggg cgggcaggtg 300
atgatgccgg ccttcgacat cggcgatgcc ggccggatgt cgttcatcac cgatccgacc 360
ggcgctgccg tgggcctatg gcaggccaat cggcacatcg gagcgacgtt ggtcaacgag 420
acgggcacgc tcatctggaa cgaactgctc acggacaagc cggatttggc gctagcgttc 480
tacgaggctg tggttggcct cacccactcg agcatggaga tagctgcggg ccagaactat 540
cgggtgctca aggccggcga cgcggaagtc ggcggctgta tggaaccgcc gatgcccggc 600
gtgccgaatc attggcacgt ctactttgcg gtggatgacg ccgacgccac ggcggccaaa 660
gccgccgcag cgggcggcca ggtcattgcg gaaccggctg acattccgtc ggtgggccgg 720
ttcgccgtgt tgtccgatcc gcagggcgcg atcttcagtg tgttgaagcc cgcaccgcag 780
caatag 786

This exemplary nucleic acid molecule, Rv0577 herein, is a tubercle-complex specific gene that was cloned and isolated from Mycobacterium tuberculosis (Mtb) H37Rv. Rv0577 has been identified as a 786 nucleotide cDNA, encoding a protein of 261 amino acids (plus the stop codon). Also suitable in the nucleic acid construct of the present invention is a nucleic acid molecule having a nucleotide sequence that hybridizes to the nucleic acid molecule corresponding to SEQ ID NO: 1 under stringent conditions. For the purposes of defining the level of stringency, reference can conveniently be made to Sambrook et al., Molecular Cloning: A Laboratory Manual, Third Edition, Cold Spring Harbor: Cold Spring Harbor Laboratory Press, New York (2001)(which is hereby incorporated by reference in its entirety). An example of high stringency conditions includes 4-5×SSC/0.1% w/v SDS at a temperature of at least 54° C. for 1-3 hours. Another stringent hybridization condition is hybridization at 4×SSC at 65° C., followed by a washing in 0.1×SSC at 65° C. for about one hour. Alternatively, an exemplary stringent hybridization condition is in 50% formamide, 4×SSC, at 42° C. Still another example of stringent conditions include hybridization at 62° C. in 6×SSC, 0.05X BLOTTO, and washing at 2×SSC, 0.1% SDS at 62° C. The skilled artisan is aware of various parameters which may be altered during hybridization and washing and which will either maintain or change the stringency conditions.

Also suitable in the nucleic acid construct of the present invention is a nucleic acid molecule having a nucleotide sequence where the nucleic acid molecule is at least 55% similar to the nucleotide sequence of SEQ ID NO: 1 by basic BLAST using default parameters analysis (Altschul et al., “Gapped BLAST and PSI-BLAST: a New Generation of Protein Database Search Programs,” Nucleic Acids Res. 25:3389-3402 (1997), which is hereby incorporated by reference in its entirety).

In one aspect of the present invention, the nucleic acid construct of the present invention has an nucleic acid molecule that encodes a protein or polypeptide having an amino acid sequence corresponding to SEQ ID NO:2, as follows:

```
Met Pro Lys Arg Ser Glu Tyr Arg Gln Gly Thr Pro Asn Trp Val Asp
    1 5 10 15
Leu Gln Thr Thr Asp Gln Ser Ala Ala Lys Lys Phe Tyr Thr Ser Leu
                          20 25 30
Phe Gly Trp Gly Tyr Asp Asp Asn Pro Val Pro Gly Gly Gly Gly Val
                  35 40 45
Tyr Ser Met Ala Thr Leu Asn Gly Glu Ala Val Ala Ala Ile Ala Pro
          50 55 60
Met Pro Pro Gly Ala Pro Glu Gly Met Pro Pro Ile Trp Asn Thr Tyr
  65 70 75 80
Ile Ala Val Asp Asp Val Asp Ala Val Val Asp Lys Val Val Pro Gly
                                  85 90 95
Gly Gly Gln Val Met Met Pro Ala Phe Asp Ile Gly Asp Ala Gly Arg
                        100 105 110
Met Ser Phe Ile Thr Asp Pro Thr Gly Ala Ala Val Gly Leu Trp Gln
                115 120 125
Ala Asn Arg His Ile Gly Ala Thr Leu Val Asn Glu Thr Gly Thr Leu
        130 135 140
Ile Trp Asn Glu Leu Leu Thr Asp Lys Pro Asp Leu Ala Leu Ala Phe
145 150 155 160
Tyr Glu Ala Val Val Gly Leu Thr His Ser Ser Met Glu Ile Ala Ala
                                165 170 175
Gly Gln Asn Tyr Arg Val Leu Lys Ala Gly Asp Ala Glu Val Gly Gly
                        180 185 190
Cys Met Glu Pro Pro Met Pro Gly Val Pro Asn His Trp His Val Tyr
                195 200 205
Phe Ala Val Asp Asp Ala Asp Ala Thr Ala Ala Lys Ala Ala Ala Ala
        210 215 220
Gly Gly Gln Val Ile Ala Glu Pro Ala Asp Ile Pro Ser Val Gly Arg
225 230 235 240
Phe Ala Val Leu Ser Asp Pro Gln Gly Ala Ile Phe Ser Val Leu Lys
                                245 250 255
Pro Ala Pro Gln Gln
                        260
```

The Rv0577 protein or polypeptide, termed Rv0577 or CFP32 herein, is the ˜32 kDa, IL-10 producing protein encoded by Rv0577 (cfp32).

The protein or polypeptide of the present invention is preferably produced in purified form by conventional techniques. Typically, the protein or polypeptide of the present invention is secreted into the growth medium of recombinant E. coli. To isolate the protein, the E. coli host cell carrying a recombinant plasmid is propagated, homogenized, and the homogenate is centrifuged to remove bacterial debris. The supernatant is then subjected to sequential ammonium sulfate precipitation. The fraction containing the protein of the present invention is subjected to gel filtration in an appropriately sized dextran or polyacrylamide column to separate the proteins. If necessary, the protein fraction may be further purified by HPLC. Alternative methods may be used as suitable. Mutations or variants of the above polypeptide or protein are encompassed by the present invention.

Variants may be modified by, for example, the deletion or addition of amino acids that have minimal influence on the properties, secondary structure, and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

Fragments of the above protein are also encompassed in this and all aspects of the present invention. Suitable fragments of the CFP32 protein having SEQ ID NO: 2 include, without limitation, those polypeptides shown in Table 1, below.

TABLE 1

| | | | |
|---|---|---|---|
| Peptide | Amino | SEQ | |
| No. | acids | ID NO: | Peptide Sequence |

| | | | |
|---|---|---|---|
| 1 | 1-25 | 32 | MPKRSEYRQGTPNWVDLQTTDQSAA |
| 2 | 21-45 | 34 | DQSAAKKFYTSLFGWGYDDNPVPGG |
| 3 | 41-65 | 36 | PVPGGGGVYSMATLNGEAVAAIAPM |
| 4 | 61-85 | 38 | AIAPMPPGAPEGMPPIWNTYIAVDD |
| 5 | 81-105 | 40 | IAVDDVDAVVDKVVPGGGQVMMPAF |
| 6 | 101-125 | 42 | MMPAFDIGDAGRMSFITDPTGAAVG |
| 7 | 121-145 | 44 | GAAVGLWQANRHIGATLVNETGTLI |
| 8a | 141-165 | 46 | TGTLIWNELLTDKPDLALAFYEAVV |
| 9 | 181-205 | 48 | RVLKAGDAEVGGCMEPPMPGVPNHW |
| 10 | 201-225 | 50 | VPNHWHVYFAVDDADATAAKAAAAG |
| 11 | 221-245 | 52 | AAAAGGQVIAEPADIPSVGRFAVLS |
| 12 | 241-261 | 54 | FAVLSDPQGAIFSVLKPAPQQ |

The nucleotide sequence corresponding to each of the exemplary P32 fragments are shown in Table 2, below.

TABLE 2

| | | |
|---|---|---|
| Peptide No. | SEQ ID NO: | Nucleotide Sequence |

| | | |
|---|---|---|
| 1 | 31 | atgcccaaga gaagcgaata |
| | | caggcaaggc acgccgaact |
| | | gggtcgacct tcagaccacc |
| | | gatcagtccg ccgcc |
| 2 | 33 | gatcagtccg ccgccaaaaa |
| | | gttctacaca tcgttgttcg |
| | | gctggggtta cgacgacaac |
| | | ccggtccccg gaggc |
| 3 | 35 | ccggtccccg gaggcggtgg |
| | | ggtctattcc atggccacgc |
| | | tgaacggcga agccgtggcc |
| | | gccatcgcac cgatg |
| 4 | 37 | gccatcgcac cgatgccccc |
| | | gggtgcaccg gaggggatgc |
| | | cgccgatctg gaacacctat |
| | | atcgcggtgg acgac |
| 5 | 39 | atcgcggtgg acgacgtcga |
| | | tgcggtggtg gacaaggtgg |
| | | tgcccggggg cgggcaggtg |
| | | atgatgccgg ccttc |
| 6 | 41 | atgatgccgg ccttcgacat |
| | | cggcgatgcc ggccggatgt |
| | | cgttcatcac cgatccgacc |
| | | ggcgctgccg tgggc |
| 7 | 43 | ggcgctgccg tgggcctatg |
| | | gcaggccaat cggcacatcg |
| | | gagcgacgtt ggtcaacgag |
| | | acgggcacgc tcatc |
| 8a | 45 | acgggcacgc tcatctggaa |
| | | cgaactgctc acggacaagc |
| | | cggatttggc gctagcgttc |
| | | tacgaggctg tggtt |
| 9 | 47 | cgggtgctca aggccggcga |
| | | cgcggaagtc ggcggctgta |
| | | tggaaccgcc gatgcccggc |
| | | gtgccgaatc attgg |
| 10 | 49 | gtgccgaatc attggcacgt |
| | | ctactttgcg gtggatgacg |
| | | ccgacgccac ggcggccaaa |
| | | gccgccgcag cgggc |
| 11 | 51 | gccgccgcag cgggcggcca |
| | | ggtcattgcg gaaccggctg |
| | | acattccgtc ggtgggccgg |
| | | ttcgccgtgt tgtcc |
| 12 | 53 | ttcgccgtgt tgtccgatcc |
| | | gcagggcgcg atcttcagtg |
| | | tgttgaagcc cgcaccgcag caa |

Suitable polypeptide fragments of the CFP32 protein of the present invention can be produced by several means. In the first, subclones of the gene encoding the protein of the present invention are produced by conventional molecular genetic manipulation by subcloning gene fragments. The subclones then are expressed in vitro or in vivo in bacterial cells to yield a smaller protein or peptide.

In another approach, based on knowledge of the primary structure of the protein of the present invention, fragments of the gene of the present invention may be synthesized by using the PCR technique together with specific sets of primers chosen to represent particular portions of the protein. These then would be cloned into an appropriate vector for increased expression of an accessory peptide or protein.

Chemical synthesis can also be used to make suitable fragments. Such a synthesis is carried out using known amino acid sequence for the protein of the present invention. These fragments can then be separated by conventional procedures (e.g., chromatography, SDS-PAGE) and used in the methods of the present invention.

The making of a nucleic acid construct of the present invention generally involves first inserting the desired nucleic acid molecule into an expression system to which the nucleic acid molecule is heterologous (i.e., not normally present). The heterologous nucleic acid molecule is inserted into the expression system which includes the necessary elements for the transcription and translation of the inserted protein coding sequences.

The nucleic acid molecule(s) of the present invention may be inserted into any of the many available expression vectors using reagents that are well known in the art. In preparing the nucleic acid constructs of the present invention, the various nucleic acid molecules of the present invention may be inserted or substituted into a bacterial plasmid-vector. Any convenient plasmid may be employed, which will be characterized by having a bacterial replication system, a marker which allows for selection in a bacterium and generally one or more unique, conveniently located restriction sites. Numerous plasmids, referred to as transformation vectors, are available for transformation. Suitable vectors include, but are not limited to, the following: viral vectors, such as lambda vector system gt11, gt WES.tB, Charon 4, and plasmid vectors such as pBR322, pBR325, pACYC177, pACYC1084, pUC8, pUC9, pUC18, pUC19, pLG339, pR290, pKC37, pKC101, SV 40, pBluescript II SK± or KS± (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology vol. 185 (1990), which is hereby incorporated by reference in its entirety), and any derivatives thereof. Also suitable are yeast expression vectors, which have been shown to be highly useful for cloning, expression, and high-throughput applications (e.g., “Holz et al., “Establishing the Yeast Saccharomyces cerevisiae as a System for Expression of Human Proteins on a Proteome Scale,” J. Functional Genomics 4(2-3):97-108 (2003); Cheng et al., “Cloning and Expression of the Gene of Augmenter of Liver Regeneration in Yeast Cells, “Hepatobiliary Pancreat Dis Int 1(1):87-91 (2002); Harger et al., “An in vivo Dual—Luciferase Assay System for Studying Translational Recoding in the Yeast Saccharomyces cerevisiae,” RNA 9:1019-1024 (2003), which are hereby incorporated by reference in their entirety). Exemplary yeast plasmids include, without limitation, pPICZ, pFLD, and PFLDα (Invitrogen, Carlsbad, Calif.), which can be inserted into a strain of P. pastoris using appropriate selection markers. The selection of a vector will depend on the preferred transformation technique and target cells for transfection.

Certain “control elements” or “regulatory sequences” are also incorporated into the plasmid-vector constructs of the present invention. These include non-transcribed regions of the vector and 5′ and 3′ untranslated regions, which interact with host cellular proteins to carry out transcription and translation. Such elements may vary in their strength and specificity. Depending on the vector system and host utilized, any number of suitable transcription and/or translation elements, including constitutive, inducible, and repressible promoters, as well as minimal 5′ promoter elements may be used.

A constitutive promoter is a promoter that directs constant expression of a gene in a cell. Examples of some constitutive promoters that are widely used for inducing expression of transgenes include the nopoline synthase (“NOS”) gene promoter, from Agrobacterium tumefaciens (U.S. Pat. No. 5,034,322 issued to Rogers et al., which is hereby incorporated by reference in its entirety), the cauliflower mosaic virus (“CaMV”) 35S and 19S promoters (U.S. Pat. No. 5,352,605 issued to Fraley et al., which is hereby incorporated by reference in its entirety), those derived from any of the several actin genes, which are known to be expressed in most cells types (U.S. Pat. No. 6,002,068 issued to Privalle et al., which is hereby incorporated by reference in its entirety), and the ubiquitin promoter (“ubi”), which is the promoter of a gene product known to accumulate in many cell types. Examples of constitutive promoters for use in mammalian cells include the RSV promoter derived from Rous sarcoma virus, the CMV promoter derived from cytomegalovirus, β-actin and other actin promoters, and the EF1α promoter derived from the cellular elongation factor 1α gene.

Also suitable as a promoter in the plasmids of the present invention is a promoter that allows for external control over the regulation of gene expression. One way to regulate the amount and the timing of gene expression is to use an inducible promoter. Unlike a constitutive promoter, an inducible promoter is not always optimally active. An inducible promoter is capable of directly or indirectly activating transcription of one or more DNA sequences or genes in response to an inducer. Some inducible promoters are activated by physical means such as the heat shock promoter (“Hsp”). Others are activated by a chemical, for example, IPTG or tetracycline (“Tet on” system). Other examples of inducible promoters include the metallothionine promoter, which is activated by heavy metal ions, and hormone-responsive promoters, which are activated by treatment of certain hormones. In the absence of an inducer, the nucleic acid sequences or genes under the control of the inducible promoter will not be transcribed or will only be minimally transcribed. When any plasmids of the present invention contain an inducible promoter, the method of the present invention further includes the step of adding an appropriate inducing agent to the cell culture when activation of the promoter is desired. Promoters of the nucleic acid construct of the present invention may be either homologous (derived from the same species as the host cell) or heterologous (derived from a different species than the host cell).

The nucleic acid molecule of the present invention, a promoter molecule of choice, a suitable 3′ regulatory region, and if desired, a reporter gene, are incorporated into a vector-expression system of choice to prepare the nucleic acid construct of present invention using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety, and U.S. Pat. No. 4,237,224 to Cohen and Boyer, which is hereby incorporated by reference in its entirety, which describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

In one aspect of the present invention, a nucleic acid molecule encoding a protein of choice is inserted into a vector in the sense (i.e., 5′→3′) direction, such that the open reading frame is properly oriented for the expression of the encoded protein under the control of a promoter of choice. Single or multiple nucleic acids may be ligated into an appropriate vector in this way, under the control of a suitable promoters, to prepare a nucleic acid construct of the present invention. In another aspect, the nucleic acid molecule is inserted into the expression system or vector in the antisense (i.e., 3′→5′) orientation. The antisense form of the nucleic acid molecule is complementary to the Rv0577 nucleic acid molecule of the present invention, or complementary to a fragment of the Rv0577 nucleic acid molecule.

Once the nucleic acid construct of the present invention has been prepared, it is ready to be incorporated into a host cell. Accordingly, another aspect of the present invention relates to a recombinant cell, or “host” containing the nucleic acid construct of the present invention. Basically, this is carried out by transforming or transfecting a host or cell with a plasmid construct of the present invention, using standard procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety. Suitable hosts and cells for the present invention include, without limitation, bacterial cells, virus, yeast cells, plant cells, and mammalian cells, including human cells, as well as any other cell system that is suitable for producing a recombinant protein. Exemplary bacterial cells include, without limitation, E. coli and Mycobacterium spp., including M. smegmatis. M. africanum, M. microti, and preferably, M. bovis Bacillus Calmette-Guerin (BCG). Exemplary yeast hosts include without limitation, Pischia pastoris and Saccharomyces cerevisiae. Methods of transformation or transfection may result in transient or stable expression of the genes of interest contained in the plasmids. After transformation, the transformed host cells can be selected and expanded in suitable culture. Preferably, transformed cells are first identified using a selection marker simultaneously introduced into the host cells along with the nucleic acid construct of the present invention. Suitable markers include markers encoding for antibiotic resistance, such as the nptII gene which confers kanamycin resistance (Fraley, et al., Proc. Natl. Acad. Sci. USA, 80:4803-4807 (1983), which is hereby incorporated by reference in its entirety), or gentamycin, G418, ampicillin, hygromycin, streptomycin, spectinomycin, tetracycline, chloramphenicol, and the like (Southern and Berg, “Transformation of Mammalian Cells to Antibiotic Resistance With a Bacterial Gene Under the Control of the SV40 Early Region Promoter,” J Mol Appl Genet., 1(4):327-41 (1982); Bernard et al., “Construction of a Fusion Gene That Confers Resistance Against Hygromycin B to Mammalian Cells in Culture,” Exp Cell Res. 158(1):237-43 (1985), which are hereby incorporated by reference in their entirety). A number of antibiotic-resistance markers are known in the art and others are continually being identified. Any known antibiotic-resistance marker can be used to transform and select transformed host cells in accordance with the present invention. Cells or tissues are grown on a selection medium containing an antibiotic, whereby generally only those transformants expressing the antibiotic resistance marker continue-to grow. Additionally, or in the alternative, reporter genes, including, but not limited to, β-Glucuronidase, luciferase, green fluorescent protein (GFP) or enhanced green fluorescent protein (EGFP), may be used for selection of transformed cells. The selection marker employed will depend on the target species; for certain target species, different antibiotics, or biosynthesis selection markers are preferable.

The present invention also relates to an isolated antibody, or binding portion thereof, against a protein or polypeptide having an amino acid sequence corresponding to SEQ ID NO: 2 of the present invention. This aspect of the present invention involves producing antibodies against the polypeptide or protein of the present invention that are capable of inhibiting the activity of a polypeptide or protein of the present invention. The antibodies of the present invention may be monoclonal or polyclonal. Monoclonal antibody production may be effected by techniques which are well-known in the art. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, “Continuous Culture of Fused Cells Secreting Antibody of Predefined Specificity,” Nature, 256:495-7 (1975), which is hereby incorporated by reference in its entirety.

Mammalian lymphocytes are immunized by in vivo immunization of the animal (e.g., a mouse) with the protein or polypeptide of the present invention. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents (Milstein et al., “Derivation of Specific Antibody-Producing Tissue Culture and Tumor Lines by Cell Fusion,” Eur. J. Immunol., 6:511-19 (1976), which is hereby incorporated by reference in its entirety). This immortal cell line, which may be derived from cells of any mammalian species, including, but not limited to, mouse, rat, and human, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering the protein or polypeptide of the present invention subcutaneously to New Zealand white rabbits which have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in E. Harlow, et. al., Editors, Antibodies: a Laboratory Manual (1988), which is hereby incorporated by reference in its entirety.

Another aspect of the present invention is a method for detection of Mycobacterium tuberculosis specific antibodies in a sample of tissue or body fluids. This method involves providing the isolated protein or polypeptide of the present invention as an antigen; contacting the sample with the antigen; contacting the sample with the antigen under conditions effective to allow formation of a complex of the antigen bound to antibodies which recognize the antigen; and detecting if any of the complex is present, thereby indicating a presence of Mycobacterium tuberculosis the sample. Body fluids suitable for this aspect of the present invention include blood, saliva, sputum, and pulmonary lavage fluid. In this aspect of the present invention, the protein or polypeptide may have a label to permit detection of binding of the antibody in a biological sample, including a tissue or body fluid. Suitable labels include a fluorescent label, a radioactive label, a nuclear magnetic resonance active label, a luminescent label, and a chromophore label. Any assay system capable of detecting a complex of the antigen bound to antibodies which recognize the antigen is suitable for this aspect of the present invention, including, but not limited to, an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay.

The present invention also relates to another method for detection of Mycobacterium tuberculosis in a sample of tissue or body fluids. This method involves providing an antibody or binding portion thereof against the protein or polypeptide of the present invention, contacting the sample with the antibody or binding portion thereof under conditions effective to allow formation of a complex of the antibody or binding portion thereof and an antigen recognized by the antibody or binding portion thereof, and detecting if any of the complex is present, thereby indicating the presence of Mycobacterium tuberculosis in the sample. As indicated above, antibodies suitable for use in accordance with the present invention include monoclonal or polyclonal antibodies. In addition, antibody fragments, half-antibodies, hybrid derivatives, probes, and other molecular constructs may be utilized. Also suitable in this aspect of the present invention are binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Academic Press 1983), which is hereby incorporated by reference in its entirety. Detecting may be carried out by any assay system capable of detecting a complex of the antibody or binding portion thereof and an antigen recognized by the antibody or binding portion, including, but not limited to, those described supra. The antibody or binding portion thereof may be labeled as describe supra, for use in a suitable assay system.

The present invention also relates to a third method for detection of Mycobacterium tuberculosis in a sample of tissue or body fluids. This method involves providing a nucleic acid molecule of the present invention as a probe in a nucleic acid hybridization assay; contacting the sample with the probe under conditions effective to permit formation of a complex of the probe and nucleic acid which hybridizes to the probe; and detecting formation of the complex in the sample thereby indicating a presence of Mycobacterium tuberculosis in the sample. Methods of detection may include, but are not limited to, electrophoresis, DNA sequencing, blotting, and in-situ hybridization.

The present invention also relates to a fourth method of detection of Mycobacterium tuberculosis in a sample of tissue or body fluids. This method involves providing a nucleic acid molecule of the present invention as a probe or primer in a gene amplification detection procedure, contacting the sample with the probe or primer under conditions effective to amplify probe or primer-specific nucleic acid molecules, and detecting any amplified probe or primer-specific molecules, thereby indicating a presence of Mycobacterium tuberculosis in the sample. A number of methods can be used to amplify the nucleic acid molecule encoding the protein of the present invention. These include, but are not limited to, polymerase chain reaction (PCR), ligase chain reaction (LCR), ligase detection reaction (LDR), LDR-PCR, strand displacement amplification, hybridization signal amplification (HSAM), self-sustained sequence (3SR) replication, Q-beta replicase, nucleic acid sequence based amplification (“NASBA”), transcription-based amplification System (“TAS”), or branched-DNA methods. Detection methods include any methods commonly associated with the method of amplification carried out, including, but not limited to, gel electrophoresis, array-capture, and direct sequencing. The nucleic acid probes in this aspect of the present invention can be labeled or tagged in accordance with the detection method of choice.

The present invention also relates to a method of vaccinating a mammal against infection by Mycobacterium tuberculosis. In one aspect of the present invention this method involves administering an effective amount of the isolated protein or polypeptide having an amino acid sequence corresponding to SEQ ID NO: 2 of the present invention to the mammal. Also suitable in this aspect are polypeptides having an amino acid sequence corresponding to SEQ ID NO: 34, 40, or, preferably, 44. In this and all other aspects of the present invention “administering” may be oral, intradermal, intramuscular, intraperitoneal, intravenous, subcutaneous, or intranasal. Oral immunization offers certain advantages over other routes of vaccination. For example, oral vaccines are more easily administered and, therefore, may be more acceptable to vaccine recipients. Also, oral vaccines can be less pure than vaccines formulated for injection, making production costs lower. Oral vaccines may also include flavorings, colorings, and other food additives to make the vaccine more palatable. In addition, oral vaccines may also contain stabilizers and preservatives to extend the shelf life of the vaccine.

The proteins or polypeptides which are to be administered as vaccines according to the present invention can be formulated according to conventional and/or future methods for such administration to the subject to be protected and can be mixed with conventional adjuvants. The peptide expressed can be used as an immunogen in subunit vaccine formulations, which may be multivalent. The product may be purified for purposes of vaccine formulation from any vector/host systems that express the heterologous protein. The purified protein or polypeptide of the present invention should be adjusted to an appropriate concentration, formulated with any suitable vaccine adjuvant and packaged for use. Suitable adjuvants include, but are not limited to: mineral gels, e.g., aluminum hydroxide; surface active substances such as lysolecithin, pluronic polyols; polyanions; peptides; oil emulsions; and potentially useful human adjuvants such as BCG (Bacille Calmette-Guerin) and Corynebacterium parvum. The immunogen may also be incorporated into liposomes, or conjugated to polysaccharides and/or other polymers for use in a vaccine formulation.

Another aspect of the present invention is a vaccine for preventing infection and disease of mammals by Mycobacterium tuberculosis. This vaccine includes an isolated protein or polypeptide having an amino acid sequence corresponding to SEQ ID NO: 2, 34, 40, or 44, of the present invention, or a combination thereof, and a pharmaceutically-acceptable carrier.

The present invention also relates to another method of vaccinating mammals against infection by Mycobacterium tuberculosis. This involves administering an effective amount of a vaccine of the present invention, made as described herein above, to mammals.

The present invention also relates to a method of treating mammals infected with Mycobacterium tuberculosis. This method involves administering an effective amount of the antibody, or a binding portion thereof, against the protein or polypeptide of the present invention to mammals infected with Mycobacterium tuberculosis. A suitable antibody of this aspect of the present invention may be a monoclonal or polyclonal antibody, or a binding portion thereof, all as described above herein. Administering of such an antibody or a binding portion thereof may be oral, intradermal, intramuscular, intraperitoneal, intravenous, subcutaneous, or intranasal. The antibodies or binding portions thereof in accordance with this and all other aspects of the present invention in which antibodies are administered to a mammal for prevention or treatment of Mycobacterium tuberculosis infection can be administered orally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes. They may be administered alone or with pharmaceutically or physiologically acceptable carriers, excipients, or stabilizers, and can be in solid or liquid form such as, tablets, capsules, powders, solutions, suspensions, or emulsions.

The solid unit dosage forms can be of the conventional type. The solid form can be a capsule, such as an ordinary gelatin type containing the antibodies or binding portions thereof of the present invention and a carrier, for example, lubricants and inert fillers such as, lactose, sucrose, or cornstarch. In another embodiment, these compounds are tableted with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The antibody or binding portion thereof of the present invention may also be administered in injectable dosages by solution or suspension of these materials in a physiologically acceptable diluent with a pharmaceutical carrier. Such carriers include sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

For use as aerosols, the antibody or binding portion thereof of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The present invention also relates to a composition for passively immunizing mammals infected with Mycobacterium tuberculosis. This composition includes an isolated antibody, or binding portion thereof, according the present invention and a pharmaceutically-acceptable carrier. A suitable antibody of this aspect of the present invention may be a monoclonal or polyclonal antibody or a binding portion thereof, prepared as described herein, and where the administration of the composition is as described herein, supra.

The present invention also relates a method of passively immunizing mammals infected with Mycobacterium tuberculosis. This method involves administering an effective amount of the composition of the present invention having the isolated antibody, or binding portion thereof, according the present invention and a pharmaceutically-acceptable carrier to mammals infected with Mycobacterium tuberculosis. Suitable antibodies and the administration thereof are as described herein, supra.

Another aspect of the present invention relates to a method of enhancing vaccination against Mycobacterium tuberculosis using a composition comprising a microorganism capable of producing an antigenic response against Mycobacterium tuberculosis when introduced into a host subject. This method involves suppressing in the microorganism the expression of a nucleic acid molecule that either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of at least 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2. Also suitable in this aspect are the nucleic acid molecules having SEQ ID NO: 33, 39, and 43, which encode polypeptides having SEQ ID NO: 34, 40, and 44, respectively. A microorganism suitable for use in this aspect is Mycobacterium bovis Bacillus Calmette-Guerin. As described in greater detail in the Examples below, the protein encoded by Rv0577 (RV0577/CFP32) is capable of suppressing the immune response in subjects infected with Mtb. Therefore, it is advantageous to interfere with the production of RV0577/CFP32 in a subject, thereby diminishing the dampening of immune response in an Mtb-infected subject. This can be carried out in a variety of ways. For example, a genetically modified microorganism for this aspect of the present invention can be prepared using the knowledge provided herein with regard to the Rv0577 gene and its protein product, in combination with conventional molecular biology techniques for manipulation of the nucleotide sequence thereof, and insertion into an appropriate expression vector for use in this aspect of the present invention (e.g., Sambrook et al., Molecular Cloning: A Laboratory Manual, Third Edition, Cold Spring Harbor: Cold Spring Harbor Laboratory Press, New York (2001); Parish et al., “glnE Is An Essential Gene in Mycobacterium tuberculosis,” J. Bacteriol. 182(20): 5715-5720 (2000), which are hereby incorporated by reference in their entirety), in accordance with common conventions for production of a composition for use in active immunization (e.g., Kuby, J., Immunology, W.H. Freeman Co., New York, N.Y. Chap. 18 (1992), which is hereby incorporated by reference in its entirety). Regulation of the expression of the nucleic acid molecule of the present invention involves transformation of a cell or tissue of choice, either in vivo or ex vivo with a suitable nucleic acid construct of the present invention. In this aspect of the present invention, in which suppression (including ablation) of expression of CFP32 is desired, this method involves preparing a recombinant mycobacterium having the CFP32-encoding nucleotide sequence (SEQ ID NO: 1) or the nucleotide sequence that encodes an active polypeptide thereof, for example, SEQ ID NO: 33, SEQ ID NO: 39, and SEQ ID NO: 43, removed from the microorganism. Alternatively, the nucleic acid construct of the present invention may be configured so that the nucleic acid molecule encodes an mRNA which is not translatable, i.e., does not result in the production of a protein or polypeptide. This is achieved, for example, by introducing into the desired nucleic acid sequence of the present invention one or more premature stop codons, adding one or more bases (except multiples of 3 bases) to displace the reading frame, and removing the translation initiation codon (U.S. Pat. No. 5,583,021 to Dougherty et al., which is hereby incorporated by reference in its entirety). This can involve the use of a primer to which a stop codon, such as TAATGA, is inserted into the sense (or “forward”) PCR-primer for amplification of the full nucleic acid, between the 5′ end of that primer, which corresponds to the appropriate restriction enzyme site of the vector into which the nucleic acid is to be inserted, and the 3′ end of the primer, which corresponds to the 5′ sequence of the enzyme-encoding nucleic acid.

Genes can be effective in the non-translatable antisense forms, as well as in the non-translatable sense form (Baulcombe, D.C., “Mechanisms of Pathogen-Derived Resistance to Viruses in Transgenic Plants,” Plant Cell 8:1833-44 (1996); Dougherty, W. G., et al., “Transgenes and Gene Suppression: Telling us Something New?” Current Opinion in Cell Biology 7:399-05 (1995); Lomonossoff, G. P., “Pathogen-Derived Resistance to Plant Viruses,” Ann. Rev. Phytopathol. 33:323-43 (1995), which are hereby incorporated by reference in their entirety).

Alternatively, a suitable construct for this aspect of the present invention includes the nucleic acid molecule of the present invention placed in a suitable vector in an antisense orientation, as described above. The use of antisense RNA to down-regulate the expression of specific genes is well known (van der Krol et al., Nature, 333:866-869 (1988) and Smith et al., Nature 334:724-726 (1988), which are hereby incorporated by reference in their entirety). Antisense nucleic acids are DNA or RNA molecules that are complementary to at least a portion of a specific mRNA molecule (Weintraub, “Antisense RNA and DNA,” Scientific American 262:40 (1990), which is hereby incorporated by reference in its entirety). Antisense methodology takes advantage of the fact that nucleic acids tend to pair with “complementary” sequences. By complementary, it is meant that polynucleotides are capable of base-pairing according to the standard Watson-Crick rules. In the target cell, the antisense nucleic acids hybridize to a target nucleic acid and interferes with transcription, and/or RNA processing, transport, translation, and/or stability. The overall effect of such interference with the target nucleic acid function is the disruption of protein expression.

The present invention also relates to another composition for actively immunizing mammals against Mycobacterium tuberculosis. This composition has a microorganism capable of producing an antigenic response against Mycobacterium tuberculosis, where the microorganism has been modified to be incapable of producing a nucleic acid molecule encoding a factor suppressing an immune response to Mycobacterium tuberculosis in a host, and a pharmaceutically-acceptable carrier. This composition involves removing or turning off the Rv0577 gene in an organism (i.e., making the gene incapable of normal transcription and/or translation) as described herein above, or as described in the art, which organism is then used for producing an antigenic response to Mtb in a mammalian. An exemplary microorganism for this aspect of the present invention is M. bovis BCG, the attenuated strain of tubercle bacillus widely used in the vaccination of humans against tuberculosis. The modification to the microorganism involves the application of standard molecular biology procedures known in the art, for example, the making of a null mutant microorganism, such as describe in Example 3 here, or those described by Sambrook et al., Molecular Cloning: A Laboratory Manual, Third Edition, Cold Spring Harbor: Cold Spring Harbor Laboratory Press, New York (2001)(which is hereby incorporated by reference in its entirety).

As described in the Background, supra, II-10 is a potent inhibitor of inflammatory response in mammals. Thus, another aspect of the present invention is a method of treating an inflammatory condition in a mammal. This method involves administering a nucleic acid construct of the present invention having a nucleic acid molecule that encodes a factor suppressing an immune response to Mycobacterium tuberculosis such that the nucleic acid molecule, when expressed in the mammal, suppresses an inflammatory response; and operably linked 5′ and 3′ regulatory elements; and administering the nucleic acid construct to a mammal under conditions effective to treat an inflammatory disease. Nucleic acid molecules suitable for this aspect of the present invention include, without limitation: a nucleic acid molecule that either: (1) has a nucleotide sequence corresponding to SEQ ID NO: 1 of the present invention, (2) has a nucleotide sequence that hybridizes to the nucleic acid corresponding to SEQ ID NO: 1 under stringent conditions characterized by a hybridization buffer comprising 5×SSC at a temperature of at least 54° C., (3) is at least 55% similar to the nucleotide sequence of SEQ ID NO: 1, 33, 39, or 43 by basic BLAST using default parameters analysis, or (4) encodes a protein or polypeptide having an amino acid sequence of SEQ ID NO: 2, 34, 40 or 44 of the present invention. “Administering” of the nucleic acid molecule in this aspect of the present invention may be oral, intradermal, intramuscular, intraperitoneal, intravenous, subcutaneous, or intranasal. This aspect of the present invention involves the treatment of inflammatory diseases including, but not limited to, bronchiectasis, asthma (Nahori et al., “Effects of Mycobacterium bovis BCG on the Development of Allergic Inflammation and Bronchial Hyperresponsiveness in Hyper-IgE BP2 Mice Vaccinated as Newborns,” Vaccine 19:1484-1495 (2001), which is hereby incorporated by reference in its entirety) and sepsis; autoimmune diseases such as lupus, rheumatoid arthritis, autoimmune arthritis, and scleroderma; inflammatory bowel diseases; multiple sclerosis; polyarteritis nodosum, temporal arteritis, and tropical spastic paralysis caused by HTLV-1.

The present invention also relates to another method of treating inflammatory disease in a mammal. This method involves providing a protein or polypeptide that suppresses an immune response to Mycobacterium tuberculosis; and administering the protein or polypeptide to a mammal under conditions effective to treat inflammatory disease. Suitable proteins or polypeptides of this aspect of the present invention are those having an amino acid sequence corresponding to SEQ ID NO: 2, 34, 40, and 44 of the present invention. Diseases that may be treated by this method include, but are not limited to, those described in the preceding paragraph.

### EXAMPLES

### Example 1

### Culture of Mycobacteria and Source of Mycobacteria Components

M. smegmatis (Ms; ATCC 23038 (lots 961 and 972) or mc²155), Mtb H37Rv (MtbRv, ATCC 27294 (lots 013, 082)), MtbH37Ra (MtbRa, ATCC 25177 (lot 082)), M. bovis Bacillus Calmette-Guerin (BCG, ATCC 27290, vaccine strain Copenhagen) were grown in Middlebrook 7H9 broth (Difco Laboratory, Detroit, Mich.) supplemented with 0.2% glycerol, 0.5% Tween 80, and 10% Middlebrook ADC (Difco Laboratory). Hygromycin B (50 μg/ml) was added for the selection of 577 null mutant or M. smegmatis transformed with pMS3 or pMS3.577 and kanamycin (25 μg/ml) was added for the selection of 577 null Mtb complemented with pMSG.577 or M. smegmatis transformed with pMSG.577. Agar cultures were grown in 7H11 medium supplemented with 0.2% glycerol, 0.5% Tween 80, and 10% Middlebrook OADC (and in some cultures with 100 μg/ml cycloheximide). Growth over 23 days in liquid medium of the parental, 577 null mutant, and complemented 577 null mutant Mtb were quantitated by absorbance at 580 nm, and by plating of serial dilutions of the broth culture onto 7H11 agar as indicated above. The initial cultures were started using frozen glycerol stocks by inoculating with approximately 2×10⁵ CFU/ml in a 30 ml supplemented Middlebrook 7H9 broth as indicated above. Mtb H37Rv components (whole cell lysate, membrane, cytosol, cell wall, culture filtrate (CFP), and CFP fractions obtained by anion exchange (QAE chromatography) and mannose capped lipoarabinomannan (manLam) were obtained from Dr. John Belisle (NIH Mtb Reagents Program, Colorado State University, Fort Collins, Colo.). Purified protein derivative (PPD) without preservatives was obtained from Adventis Pasteur (formerly Pasteur, Merieux, Connaught), Swiftwater, Pa. All Mtb reagents shown were tested for endotoxin (lipopolysaccharide, LPS) by the Limulus amebocyte assay (BioWhittaker, Inc., Walkersville, Md.) and contained less than 50 pg of LPS per μg of reagent or per 10⁶ CFU of mycobacteria.

### Example 2

### Human Monocyte Isolation, Culture, and Challenge with Mycobacteria Components

Human monocytes were purified from peripheral blood mononuclear cells (PBMC) obtained as leukocyte enriched packs (New York Blood Center, New York, N.Y.). Purification was performed by either self-agglutination (Mentzer et al., “Spontaneous Aggregation as a Mechanism for Human Monocyte Purification,” Cell. Immunol. 10(2):312-19 (1986), which is hereby incorporated by reference in its entirety) and cultured in RPMI 1640 medium supplemented with 100 g/ml streptomycin, 100 U/ml penicillin and 100 g/ml L-glutamine, or by negative selection (immunodepletion) using manufacturer's protocol (StemCell Technologies, Vancouver, Canada) and cultured in X-Vivo-20 medium (Bio-Whittaker, Wakefield, Md., an artificial medium without antibiotics, serum nor LPS). Human monocytes were stimulated with Ms, BCG, Mtb, or Mtb components on day of isolation or after 24 h in culture. Cell free supernatants obtained 48 h after challenge with mycobacteria or Mtb components and stored in −80 ° C. were assayed for cytokines (IL-10, TNF-α, or IL-1β) using commercial kits or antibody pairs as directed by the manufacturer.

### Example 3

### Construction of the 577 Null Mutant of M. tuberculosis

The 577 null mutant Mtb was derived from Mtb H37Rv using the methods described by Parish et al., “glnE Is An Essential Gene in Mycobacterium tuberculosis,” J. Bacteriol. 182(20): 5715-5720 (2000) (which is hereby incorporated by reference in its entirety), which involved four sequential cloning steps and two stage selection after transformation with the suicide vector, p2NIL/5f/hyg/3f/hyg/PacI cassette. First, the 5′ flank region of Rv0577, the 3′ flank region of Rv0577, and the hygromycin resistance marker gene were cloned into p2NIL, and transformed into competent E. coli DH5α. The 5′ flank (Sf, 2252 bp) was PCR amplified using the primer pairs: forward, 5′-ATT AAA GCT TAC CCG ACC G CGT GAC CAG CGG TC-3′ (SEQ ID NO: 3), and reverse, 5′-ATT ATC TAG AGA TCA TCC TTT CGT TAG GTG GCG-3′ (SEQ ID NO: 4), and ligated into the p2NIL vector between HindIII/XbaI creating the pNIL5f plasmid. The 3′ flank (3f, 1839 bp) was PCR amplified using primer pairs: forward, 5′-ATT ATC TAG ACA GCA ATA GGG AGC ATC CCG GG-3′ (SEQ ID NO: 5), and reverse, 5′ -ATT AGC AGC GAC GGT GTC AAC GGT TC-3′ (SEQ ID NO: 6), and ligated into the XbaI and KpnI sites of the pNIL5f plasmid creating the pNIL5f/3f plasmid. The hygromycin gene was PCR amplified from pMS2 using primer pairs: forward, 5′-ATT ATC TAG ACC CTG TGA ATA GAG GTC CGC-3′ (SEQ ID NO: 7), and reverse, 5′-ATT TCT AGA CTG GAG GAG ATG ATC GAG GAT-3′ (SEQ ID NO: 8), and ligated into the single XbaI site in the middle of 5′ and 3′ flanking sequences, creating the pNIL5f/hyg/3f plasmid. All PCR products were confirmed by DNA sequence analysis using an automated sequencer (ABI Prism 310 Genetic Analyzer, Perkin Elmer). The PacI cassette from the pGOAL17 plasmid which contains an Ag85 promoter driving the lacZ gene (P_{Ag85}-LacZ) and hsp60 driving the sacB gene (Pₕₛₚ₆₀-sacB) was excised and cloned into a single PacI site within the p2NIL/5f/hyg/3f plasmid to yield the suicide vector, p2NIL/5f/hyg/3f/hyg/PacI cassette (Parish et al., “glnE Is An Essential Gene in Mycobacterium tuberculosis,” J. Bacteriol. 182(20): 5715-5720 (2000), which is hereby incorporated by reference in its entirety). The plasmids obtained from E. coli DH5α clones were confirmed to possess the ˜11 kb fragment. The selection for 577 null mutant Mtb was as follows. Competent MtbH37Rv were transformed by electroporation with the p2NIL5f/hyg/3f/PacI vector, plated onto 7H11 plates containing hygromycin and kanamycin and incubated for 3 weeks. Mtb colonies obtained by this selection step are single crossovers. Next, single colonies were inoculated into hygromycin containing liquid medium and incubated for 2 weeks to select for the second cross-over. Lastly, growing bacteria were plated onto 7H11 agar containing hygromycin, X-gal (˜40 μg/ml) and 2% sucrose for 3 weeks to select for white colonies that have lost the PacI cassette and replacement of the Rv0577 gene by the hygromycin^{R} gene.

### Example 4

### Complementation of 577 Null Mutant M. tuberculosis

The 577 null mutant Mtb was genetically complemented by introducing a wild type copy of the Rv0577 gene using the pMSG plasmid vector. The complete coding sequence of Rv0577 gene was PCR amplified from H37Rv genomic DNA using the primer pairs: forward, 5′-ATA TTA ATT AAG ATG CCC AAG AGA AGC-3′ (SEQ ID NO: 9), and reverse, 5′- ATT GGA TCC CTA TTG CTG CGG TGC GG-3′ (SEQ ID NO: 10). The amplicon was cloned into the pMSG vector between the PacI and BamHI restriction sites. The glutamate synthase (GS) promoter PCR amplified from MtbH37Rv using the primer pairs forward (Glute5), 5′-GGA CTA GTG CGA TCA GCC AGT CGA TCA GCA GAG-3′ (SEQ ID NO: 11), and reverse (Glute3) 5′-CCT TAA TTA ATT CCG TCA CAG AAT GCT CCT TTA C-3′ (SEQ ID NO: 12), was cloned into the promoterless pMS2 vector at the SpeI and PacI site(s) generating the pMSG vector (Harth et al., “Expression and Efficient Export of Enzymatically Active Mycobacterium tuberculosis Glutamine Synthetase in Mycobacterium smegmatis and Evidence that the information for Export is Contained Within the Protein,” J. Biol. Chem. 272: 22728-22735 (1997); Kaps et al., “Energy Transfer Between Fluorescent Proteins Using a Co-Expression System in Mycobacterium smegmatis,” Gene 278: 115-124 (2001); Ehrt et al., “Reprogramming of the Macrophage Transcriptome in Response to Interferon-gamma and Mycobacterium tuberculosis: Signaling Roles of Nitric Oxide Synthase-2 and Phagocyte Oxidase, J Exp Med 194(8): 1123-40 (2001), which are hereby incorporated by reference in their entirety). In the pMSG plasmid, the Rv0577 gene under the control of the GS promoter is transcribed constitutively. The pMSG.577 plasmid purified from E. coli was electroporated into competent 577 null mutant Mtb. Transformants were obtained after plating on supplemented 7H11 plates containing 25 μg/ml kanamycin. Complementation was confirmed by Southern blot, PCR amplification, and Western blot analyses.

### Example 5

### Over-Expression of Rv0577 in M. smegmatis

The Rv0577 gene was PCR amplified with primers containing PacI and HindIII enzyme cleavage sites for subsequent cloning using the following primers: forward, 5′-CCC TTA AAT GTC CGC CAC CTA ACG AAA G-3′) (SEQ ID NO: 13) and reverse (5′-CCC AAG CTT CTA GCA TTC TCC GAA-3′ (SEQ ID NO: 14) primers which amplified the coordinates 671137 to 672002 of the full Rv genome (GenBank Accession No. NC_{—}000962). The amplicon sequenced validated as Rv0577 was cloned into pMS3 a plasmid derived from the promoterless pMS2 vector by insertion of the M. tuberculosis heat shock protein promoter (hsp60) in front of the multiple cloning sites (Kaps et al., “Energy Transfer Between Fluorescent Proteins Using a Co-Expression System in Mycobacterium smegmatis,” Gene 278: 115-124 (2001); Ehrt et al., “Reprogramming of the Macrophage Transcriptome in Response to Interferon-gamma and Mycobacterium tuberculosis: Signaling Roles of Nitric Oxide Synthase-2 and Phagocyte Oxidase,” J Exp Med 194(8):1123-40 (2001), which are hereby incorporated by reference in their entirety). The pMS3 (pMS2) plasmid also contains the hygromycin® resistance gene. The plasmids, pMS3 and pMS3.577 purified from E. coli, respectively DH5α and JM109, were transformed into competent M. smegmatis MC²155. The transformants plated on supplemented 7H11 agar containing hygromycin (50 μg/ml) were validated as transformants containing pMS3 or pMS3.577 by analysis of the amplicon size after PCR amplification.

For analysis of the genetic constructs, gene amplification by PCR utilized primer pairs to detect all mycobacteria, as follows: 16S rRNA: forward, 5′-ACG GTG GGT ACT AGG TGT GGG TTT C-3′ (SEQ ID NO: 15) and reverse, 5′-TCT GCG ATT ACT AGC GAC TCC GAC TTC A -3′ (SEQ ID NO: 16); only Mtb complex subspecies (MPB70) forward, 5′-GGC GAT CTG GTG GGC CCG -3′ (SEQ ID NO: 17), and reverse, 5′- CGC CGG AGG CAT TAG CAC GCT -3′ (SEQ ID NO: 18); only M. smegmatis (Ms0911) forward, 5′-ACG CGA AGT CGG GCA ACA C 3′ (SEQ ID NO: 19) and reverse, 5′-GCG GCA GCG GGC GGG AGC AAC T -3′ (SEQ ID NO: 20), designed using Tigr.org database); and Rv0577 (cfp32) forward, 5′-ATG CCC AAG AGA AGC GAA TAC AGG CAA-3′ (SEQ ID NO: 21), and reverse 5′ -CTA TTG CTG CGG TGC GGG CTT CAA-3′ (SEQ ID NO: 22). Additional primer pairs were as follows for: pMS3 multiple cloning sites (MCS) forward, 5′-CGA GGG GAT TAC ACA TGA CCA ACT-3′ (SEQ ID NO: 23) and reverse, 5′-CGG AAG AGC GCC CAA TAC G-3′ (SEQ ID NO: 24); pMSG kan-flag-MCS sequencing: forward, 5′-ATA ACG TTC TCG GCT CGA TGA TCC-3′ (SEQ ID NO: 25), and reverse, 5′-ATC CCC TGA TTC TGT GGA TAA CCG TAT TA-3′ (SEQ ID NO: 26); and Rv0577 sequencing forward, 5′-ACC ACC TTG TCC ACC ACC GCA T-3′ (SEQ ID NO: 27), and reverse, 5′-CGA ATC ATT GGC ACG TCT ACT TTG-3′ (SEQ ID NO: 28). The Rv0577 deletion locus of 577 Null was PCR amplified using the following primer pairs: forward 577PROF, 5′-GTG GCT TGG CGG GCA CGG TGG AG-3′ (SEQ ID NO: 29) and reverse 1Dn577R, 5′-GTG GCA CCG GCG GCA CCG CAC ACC T-3′ (SEQ ID NO: 30).

### Example 6

### Selective Induction of IL-10 in M. tuberculosis

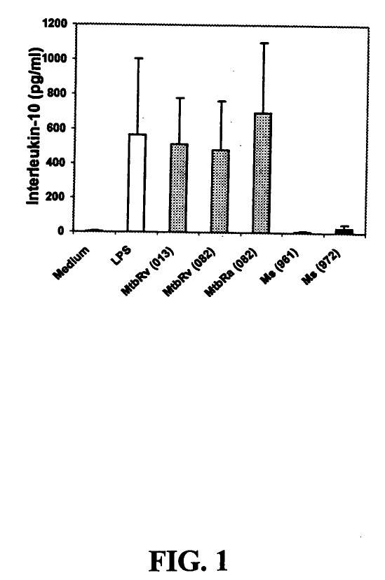

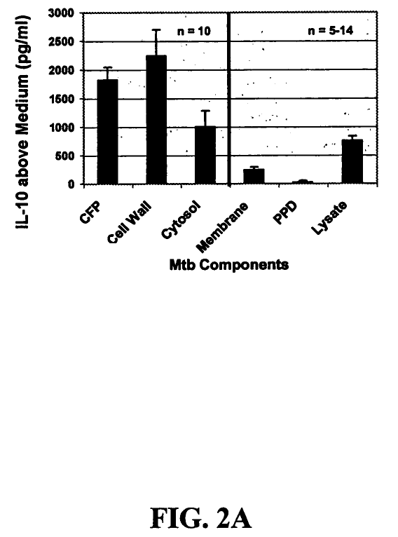

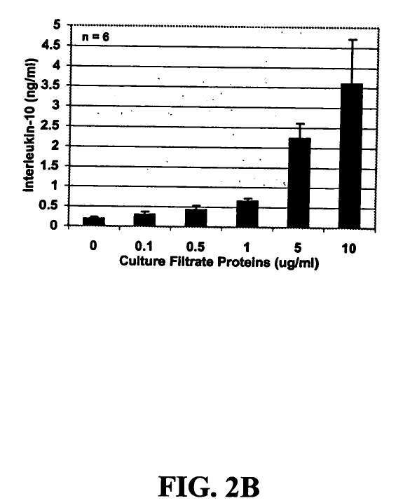

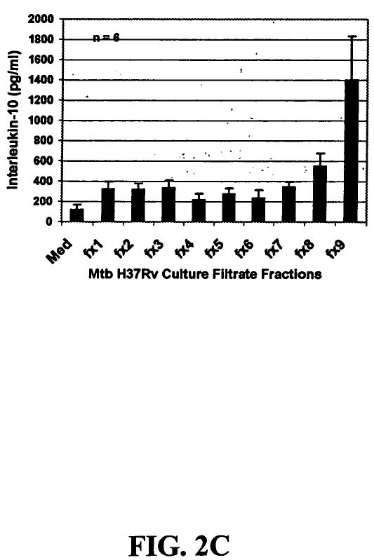

To evaluate whether IL-10 induction is restricted to Mtb, a comparison to M smegmatis, a non-pathogenic mycobacteria, was conducted. Mtb H37Rv (virulent laboratory strain) isolates and Mtb H37Ra (an attenuated strain) infection of human monocytes induced IL-10 production at levels that were similar to LPS. These results are shown in FIG. 1. M bovis BCG also induced IL-10. In contrast, two strains of M smegmatis did not induce IL-10. No reduction in monocyte viability was seen in the M smegmatis-infected monocytes at the experiments' end. To discern what component of Mtb bore IL-10 inducing activity, the whole cell lysate, culture filtrate (CFP), cell wall, cytosol, and cell membrane fractions were evaluated and compared with purified protein derivative (PPD) of Mtb. Significant IL-10 inducing activity resided in the cell wall, cytosol, and CFP and, to a lesser degree, the whole cell lysate, as shown in FIG. 2A. The CFP was of greatest interest as it is known to contain proteins that are immunogenic and are perfectly situated for immunomodulation. These properties of CFP led to its further evaluation. The stimulation of fresh monocytes by CFP to produce IL-10 was dose-dependent (from 0.1 to 10 μg/ml), as shown in FIG. 2B. Proteinase K treatment (5 ul, overnight 4° C.) of 0.1 μg CFP reduced 80% of the IL-10 inducing activity while IL-10 production by fresh monocytes treated with 0.1 μg/ml CFP was 235±39 pg/ml (n=3). IL-10 production by monocytes cultured with proteinase K or in medium alone was similarly low (n=3). As shown in FIG. 2C, the IL-10 inducing activity could be enriched by anion-exchange chromatography. Compared to 1 μg/ml CFP, anion exchange chromatography fractions (fx1 to fx9) at a concentration of 0.1 μg/ml were tested, and the highest IL-10inducing activity was seen in fx9. The IL-10induction by 0.2 μg/ml of fx9 of CFP was comparable to 5 μg/ml of whole CFP, as seen in FIG. 2B versus FIG. 2C. A prominent 32 kDa protein was seen most prominently in fx9 on silver stained gel of this fraction, and was extracted to clone CFP32, the protein encoded by Rv0577. In Table 1, below, a sandwich ELISA was used to correlate the amount of Rv0577 (expressed as pg Rv0577 per μg of reagent) with IL-10 inducing activity (expressed as pg IL-10 per μg reagent).

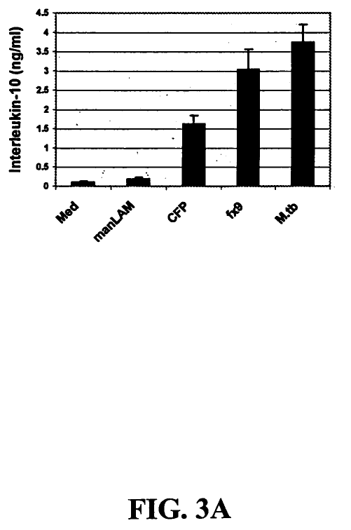

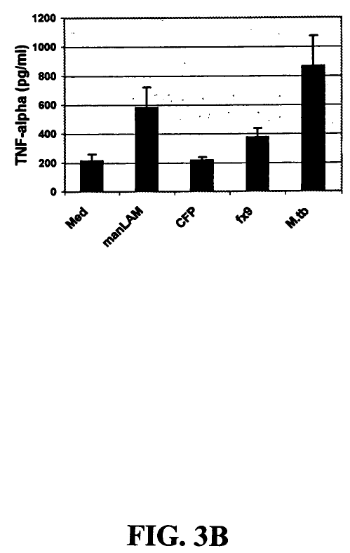

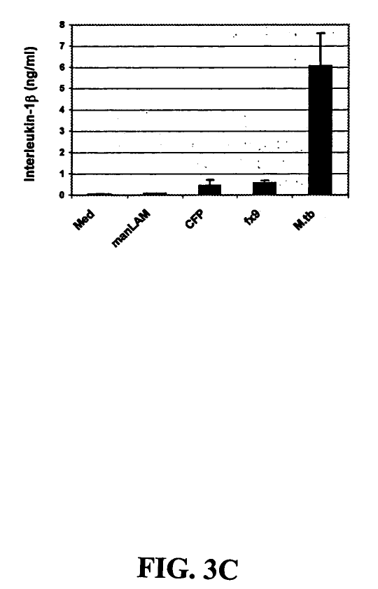

TABLE 3
Assessment of Rv0577 Protein (CFP32) in Mtb Reagents
Tested for IL-10 Inducing Activity¹

| | | IL-10 Inducing |
|---|---|---|
| | Amount of Rv0577 protein | Activity |
| Mtb reagent | (pg Rv0577/μg reagent) | (pg IL-10/μg reagent) |
| manLAM | 28 ± 21 | 82 ± 44 |
| PPD | 53 ± 37 | 51 ± 28 |
| Whole Cell Lysate | 1559 ± 378 | 759 ± 83 |
| Cytosol | 1043 ± 66 | 1008 ± 280 |
| CFP (lot1) | 663 ± 195 | 1630 ± 225 |
| Fraction 9 of CFP | 61,000 ± 5,300 | 15,210 ± 2,600 |
| (fx9) | | |
| ¹The ELISA assay for quantitation of Rv0577 protein in Mtb reagents was performed at least twice and is expressed as mean ± SD. The production of IL-10 induced by each reagent was by the following number of donors: 12, 5, 14, 10, 17 and 10, respectively. The amount of each reagent used are indicated in the legend of FIGS. 2 and 3. All Mtb reagents shown were tested for endotoxin (lipopolysaccharide, LPS) and contained less than 50 pg of LPS per μg of reagent. | | |
| # In this system, a minimum of 10 ng/ml of LPS is needed to induce IL-10; and 100 ng/ml is required to attain IL-10 levels comparable to 0.5 cfu Mtb. | | |

The data presented in Table 3 suggest a positive correlation between CFP32 and IL-10 inducing activity. Whether IL-10 is produced in concert with pro-inflammatory cytokines was examined next. In addition to IL-10, Mtb induces the simultaneous production of high levels of both IL-10 and TNF-α. The major component of Mtb cell wall, mannose capped lipoarabinomannan (manLam), induced only TNF-α. In contrast, CFP and an anion exchange chromatography fraction (fx9) of CFP induced minimal amounts of IL-1β and TNF-α, while possessing high IL-10 inducing activity, as shown in FIGS. 3A-C. These data suggest a more selective induction of cytokines by CFP and CFP fx9 of Mtb. The more selective induction of IL-10 by CFP and fx9 while Mtb and LPS simultaneously induce all three cytokines (IL-10, TNF-α, and IL-1β) argues against contamination of CFP and fx9 of Mtb by LPS or other microorganisms.

### Example 7

### M. tuberculosis Encodes a Protein Conferring IL-10 Inducing Activity

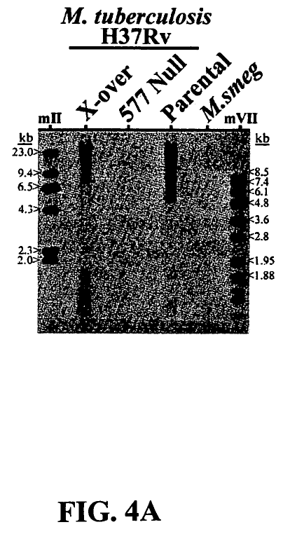

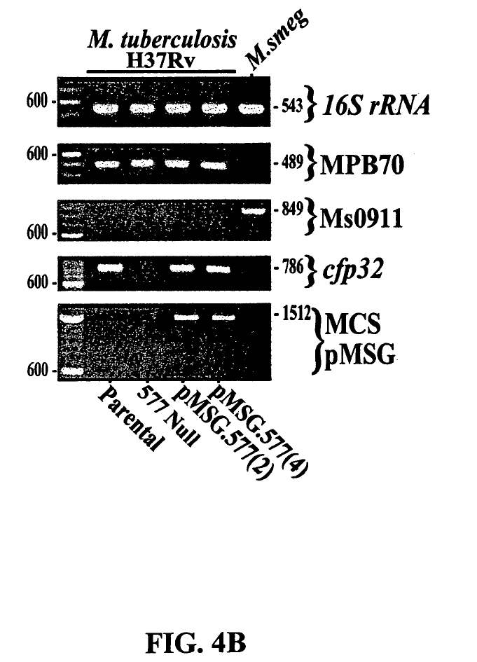

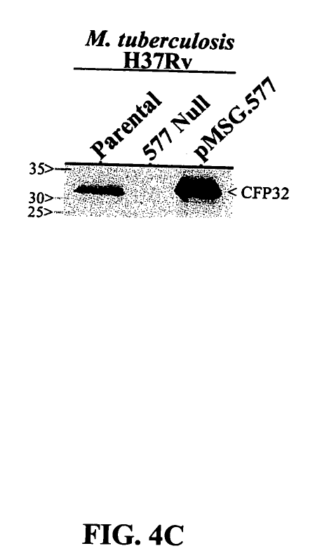

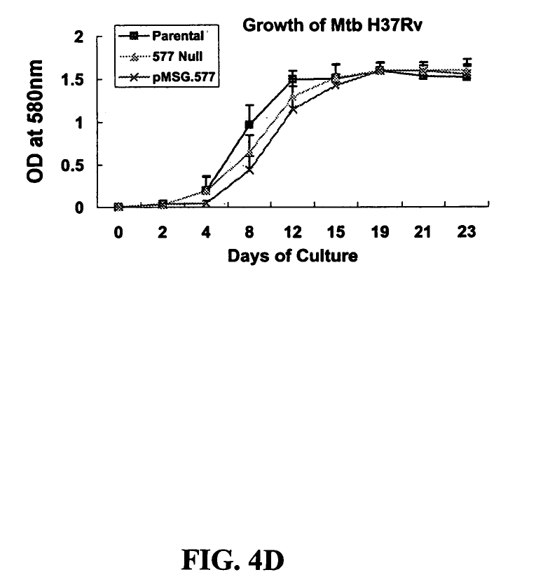

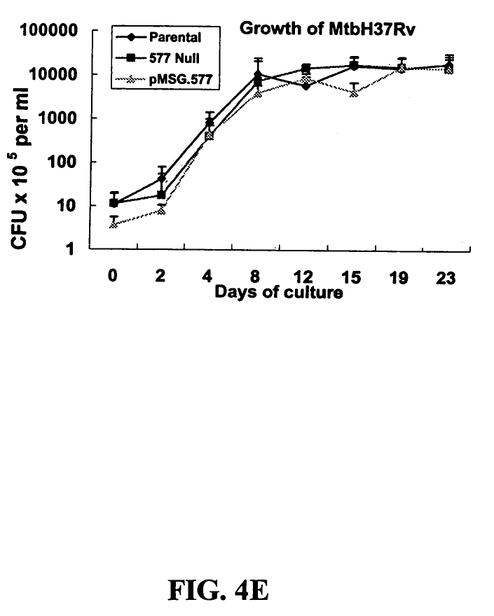

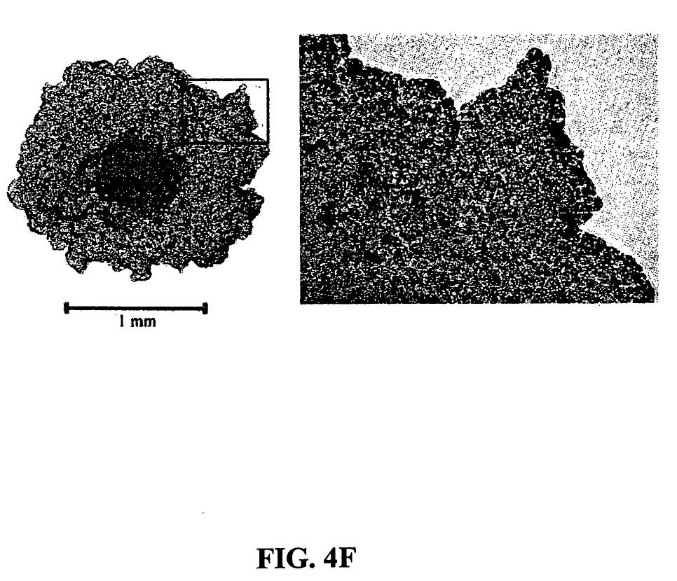

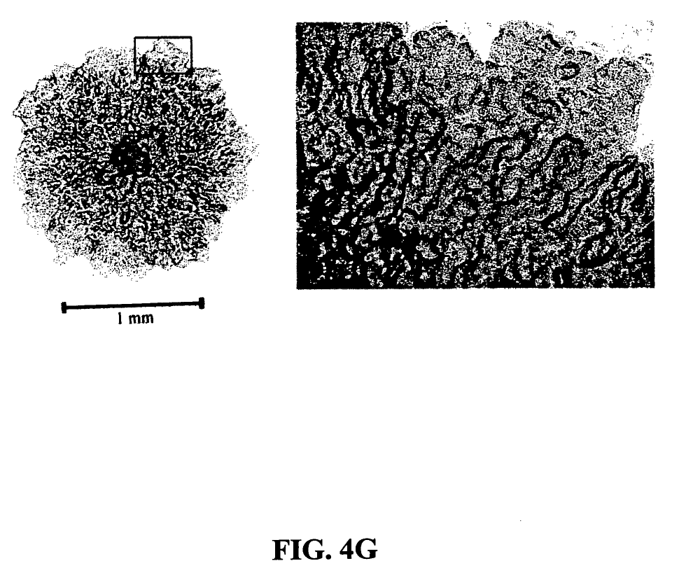

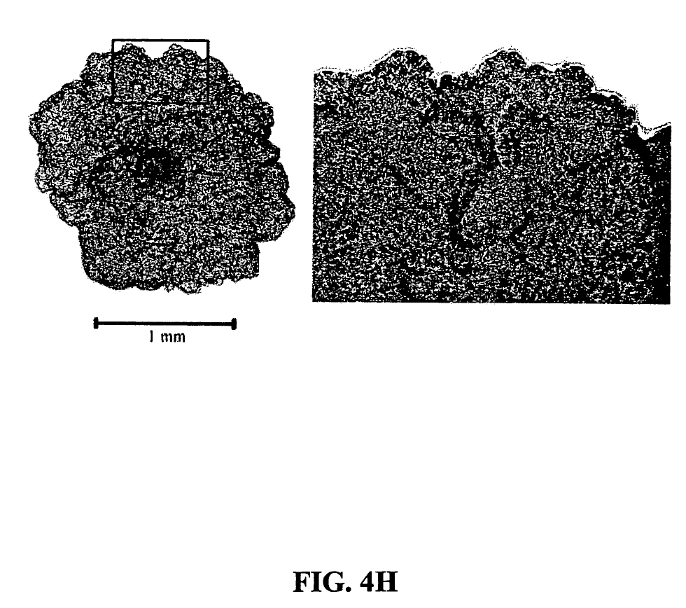

To show that Rv0577 encodes a protein that conferred IL-10 inducing activity to Mtb, a 577 null mutant was created by homologous recombination to replace Rv0577 with the hygromycin B resistance gene. Southern blot analysis demonstrated the step-wise derivation of the 577 null (double cross-over, replacement of Rv0577) from the single cross-over (X-over) which still retains the Rv0577 gene. As internal controls, the presence of Rv0577 in the parental MtbH37Rv and absence of Rv0577 in M. smegmatis are illustrated in FIG. 4A. PCR gene amplification analysis demonstrate the presence of Rv0577 within the pMSG plasmid of the complemented 577 null mutant Mtb and confirmed the loss of Rv0577 in the 577 null mutant, as shown in FIG. 4B. Using Western blot analysis, the complementation of Rv0577 with pMSG.577 plasmid under the constitutive glutamine synthase promoter was capable of restoring protein expression and the 577 null mutant lacked CFP32 expression, shown in FIG. 4C. The growth of the 577 null mutant and complemented null mutant Mtb in liquid and solid medium was examined next. Growth of the parental and mutant isolates were quantitated by absorbance measurement and by plating of individual colonies on agar. The 577 null mutant compared to parental Mtb showed similar growth kinetics, shown in FIG. 4D and FIG. 4E. Compared to parental and 577 null Mtb, the initial proliferation of the complemented 577 null mutant was slightly slower but the difference was erased by 2 weeks of culture. Evaluation of the colony morphology grown 7H11 enriched agar showed that the 577 null mutant (gene knockout), shown in FIG. 4G, differed from that of the parental (wildtype) Mtb, shown in FIG. 4F. The parental had a smooth topography and colony edges, while the Rv0577 null mutant had a much more mountainous topography and ruffled edges. This phenotype was restored by complementation with the pMSG.577 plasmid, as shown in FIG. 4H.

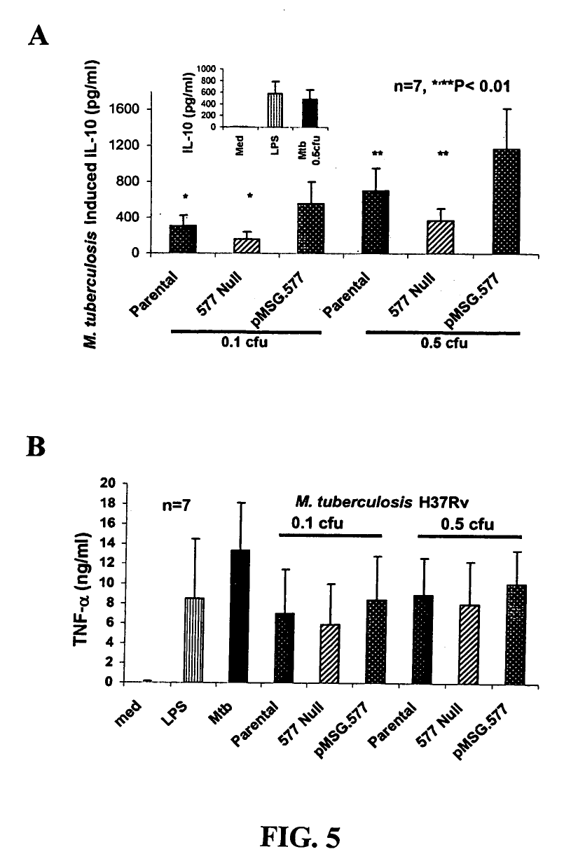

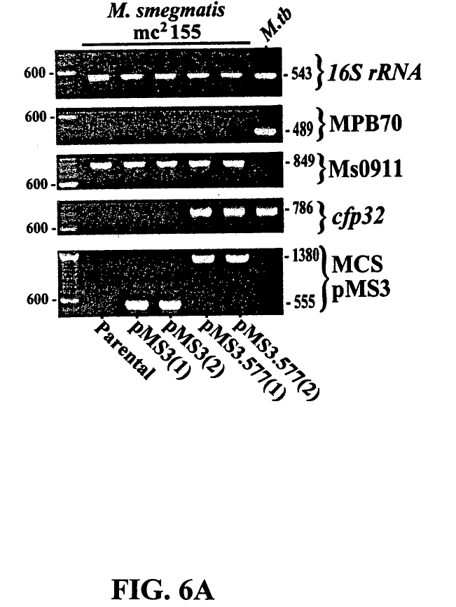

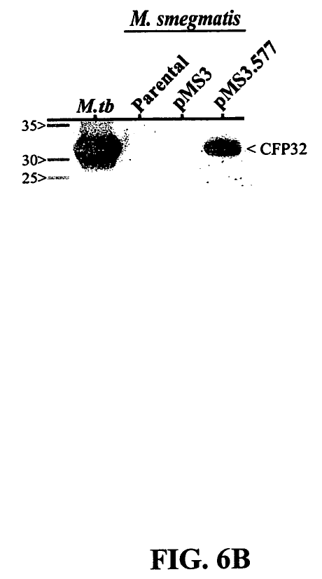

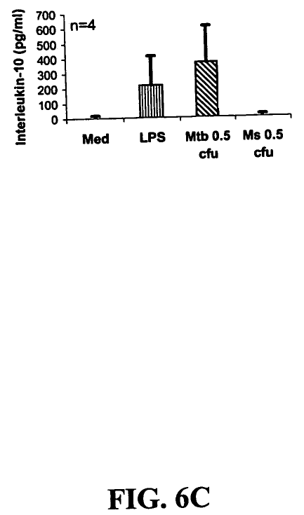

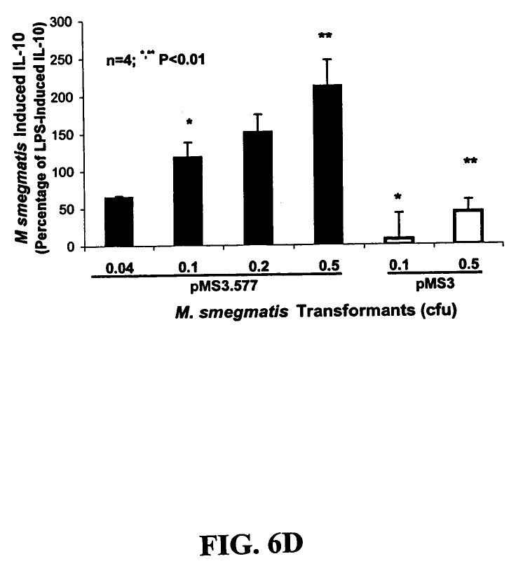

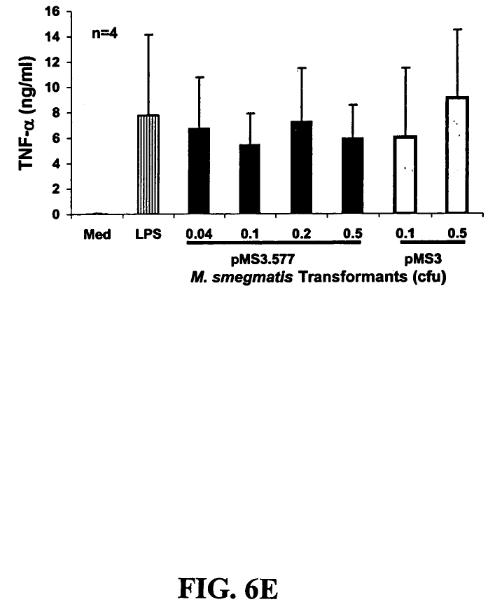

The availability of these mutants allowed the testing of whether the expression of Rv0577 is associated with IL-10 production. As illustrated in FIG. 5A, the Rv0577 null mutant compared to parental Mtb showed ≧50% reduction in IL-10 inducing activity that was statistically significant for both doses tested, 0.1 cfu/Mφ and for 0.5 cfu/Mφ (p≦0.01). Furthermore, complementation of the 577 null mutant restored IL-10 inducing activity. In contrast to IL-10, similar amounts of TNF-α were produced by monocytes infected with parental, 577 null, and complemented 577 null mutant Mtb, shown in FIG. 5B. To further validate that Rv0577 encodes a protein that conferred IL-10 inducing activity, M. smegmatis was transformed with pMS3.577 or pMS3, both expressing the hygromycin B resistance gene. Gene amplification analysis showed that M. smegmatis transformed with pMS3.577 contained the Rv0577 within the pMS3 cloning site, as shown in FIG. 6A. Furthermore, pMS3.577 M. smegmatis expressed Rv0577 protein. In contrast, neither parental M. smegmatis nor pMS3 M. smegmatis had detectable expression of Rv0577 protein, shown in FIG. 6B. As shown in FIG. 6C, parental M. smegmatis infection of human monocytes induced little to no IL-10 above medium, while LPS and MtbH37Rv, internal controls for each experiment, induced high amounts of IL-10 that showed considerable donor to donor variability. Because of the donor variability in IL-10 production, the induction of IL-10 by M. smegmatis transformants was expressed as a percentage of LPS-induced IL-10, as shown in FIG. 6D. Phenotypically, over-expression of Rv0577 by M. smegmatis led to a dose-dependent induction of IL-10 production, FIG. 6D. The induction of IL-10 is statistically significantly higher in M. smegmatis transformed with pMS3.577 than pMS3, as shown in FIG. 6D. Moreover, infection by 0.5 cfu of M. smegmatis transformed with pMS3.577 resulted in levels of IL-10 production comparable to same inoculum of Mtb, as shown in FIG. 6C versus 6D. M. smegmatis transformed with pMSG.577 (the plasmid used to complement the 577 null Mtb mutant) similarly conferred the ability to induce IL-10 (n=2). The specificity of the effect of Rv0577 protein expression on IL-10 induction is further suggested by the finding that TNF-α production was similar upon monocytes infection by either pMS3.577 or pMS3 transformed M. smegmatis, as shown in FIG. 6E.

M. tuberculosis, in contrast to M. smegmatis infection of human monocytes, triggered the production of IL-10, a potent suppressor of antimicrobial activity. The screening of the culture filtrate of Mtb H37Rv for a factor with IL-10 inducing activity identified Rv0577. Through Rv0577 gene knockout and complementation in Mtb, it was demonstrated that 577 null mutant Mtb infection of monocytes produced significantly less amounts of IL-10, and complementation of the 577 null mutant restored the expression of Rv0577 (CFP32) and IL-10 production to levels comparable to parental Mtb. From the 577 null mutant monocyte infection studies, the attributed IL-10 inducing activity to Rv0577 is in the order ˜60%. Moreover, in a heterologous mycobacteria, M. smegmatis, over-expression of Rv0577 by another plasmid resulted in an inoculum-dose dependent IL-10 production by infected monocytes. The changes observed with IL-10 production associated with the molecular manipulation of Rv0577 were specific because the simultaneous measurement of TNF-α produced by monocytes infected with M. tuberculosis or M. smegmatis mutants showed no significant differences.

The cytokine milieu as microbes encounter the innate and acquired immune cells is thought to be critical to the overall immune response and effective elimination of the invading agent (Medzhitov et al., “Innate Immunity,” New England J Med. 343(5):338-44 (2000); Sieling et al., “Toll-Like Receptors: Mammalian “Taste Receptors” For a Smorgasbord of Microbial Invaders,” Curr Opin Microbiol 5(l):70-5 (2002); Janeway et al., “Innate Immune Recognition,” Annu Rev Immunol 20:197-216 ( 2002); Fitzgerald et al., “The Role of the Interleukin-1/Toll-Like Receptor Superfamily in Inflammation and Host Defense,” Microbes Infect. 2(8):933-43 (2000); which are hereby incorporated by reference in their entirety). Manipulation of this environment is thought to be a strategy for microbial evasion and survival, and several examples have been reported. One strategy, for example, is the interference with the production of TNF-α and IFN-γ cytokines critical for innate and acquired immune cell activation to clear intracellular microbes. The Yersinia Yop gene product blocks TNF-α production by macrophages through interference with intracellular signaling molecules (Orth et al., “Disruption of Signaling by Yersinia Effector YopJ, a Ubiquitin-Like Protein Protease.,” Science 290(5496): 1594-7 (2000); Boland et al., “Role of YopP in Suppression of Tumor Necrosis Factor Alpha Release by Macrophages During Yersinia Infection,” Infect. Immun. 66(5): 1878-84 (1998); Cornelis et al., “Yersinia Lead SUMO Attack,” Nat. Med. 7:21-23 (2001), which are hereby incorporated by reference in their entirety). The adenovirus E3 14.7 Kd protein is reported to interfere with TNF-α mediated cytolytic activity, thereby blocking viral clearance (Trufariello et al., “Adenovirus E3 14.7-kDa Protein, an Antagonist of Tumor Necrosis Factor Cytolysis, Increases the Virulence of Vaccinia Virus in Severe Combined Immunodeficient Mice,” Proc. Natl. Acad. Sci. USA 91:10987-91 (1994); Trufariello et al., “Adenovirus E3 14.7-kDa Protein, an Antagonist of Tumor Necrosis Factor Cytolysis, Increases the Virulence of Vaccinia Virus in a Murine Pneumonia Model,” J. Virol. 68:453-62 (1994), which are hereby incorporated by reference in their entirety). The Mtb cell wall constituent, mannosylated lipoarabinomannans (manLAM), is reported to suppress IL-12 production, a cytokine required for T helper-1 cell maturation and secretion of IFN-γ (Nigou et al., “Mannosylated Lipoarabinomannans Inhibit IL-12 Production by Human Dendritic Cells: Evidence for a Negative Signal Delivered through the Mannose Receptor,” J. Immunol. 166(12):7477-85 (2001), which is hereby incorporated by its entirety). Encoding or inducing the production of factors to suppress host immune response is another strategy (Stockl et al., “Human Major Group Rhinoviruses Down-Modulate the Accessory Function of Monocytes by Inducing IL-10,” J. Clin. Invest. 104(7):957-65 (1999); Fleming et al., “A Homolog of Interleukin-10 is Encoded by the Poxvirus Orf Virus,” J. Virol. 71(6):4857-61 (1997); Vockerodt et al., “The Epstein-Barr Virus Latent Membrane Protein 1 Induces Interleukin-10 in Burkitt's Lymphoma Cells but not in Hodgkin's Cells Involving the p38/SAPK2 Pathway,” Virology 280(2):183-98 (2001); Henke et al., “Viral IL-10 Gene Transfer Decreases Inflammation and Cell Adhesion Molecule Expression in a Rat Model of Venous Thrombosis,” J. Immunol. 164(4):2131-41 (2000); Suzuki et al., “Viral Interleukin 10 (IL-i 0), the Human Herpes Virus 4 Cellular IL-10 Homologue, Induces Local Anergy to Allogeneic and Syngeneic Tumors,” J. Exp. Med. 182(2):477-86 (1995), which are hereby incorporated by reference in their entirety). The EBV encoded human IL-10 homolog and the EBV latent protein-10 that elicits IL-10 production both facilitate viral survival and pathogenesis through IL-10's immune suppressive activity (Vockerodt et al., “The Epstein-Barr Virus Latent Membrane Protein 1 Induces Interleukin-10 in Burkitt's Lymphoma Cells but not in Hodgkin's Cells Involving the p38/SAPK2 Pathway,” Virology 280(2): 183-98 (2001); Henke et al., “Viral IL-10 Gene Transfer Decreases Inflammation and Cell Adhesion Molecule Expression in a Rat Model of Venous Thrombosis,” J. Immunol. 164(4):2131-41 (2000); Suzuki et al., “Viral Interleukin 10 (IL-10), the Human Herpes Virus 4 Cellular IL-10 Homologue, Induces Local Anergy to Allogeneic and Syngeneic Tumors,” J. Exp. Med. 182(2):477-86 (1995), which are hereby incorporated by reference in their entirety). Induction of host IL-10 by Schistosoma eggs and HIV-1 gp41 and gp120 is thought to contribute to the immune suppression caused by these pathogens (Wynn et al., “Analysis of Granuloma Formation (by Schistomsoma eggs) in Double Cytokine-Deficient Mice Reveals a Central Role for IL-10 in Polarizing Both T Helper Cell 1- and T Helper Cell 2-Type Cytokine Responses In vivo,” J. Immunol. 159(10):5014-23 (1997); Barcova et al., “gp41 Envelope Protein of Human Immunodeficiency Virus Induces Interleukin (IL)-10 in Monocytes, but not in B, T, or NK Cells, Leading to Reduced IL-2 and Interferon-Gamma Production,” J. Infect. Dis. 177(4):905-13 (1998); Taoufik et al., “Human Immunodeficiency Virus gp120 Inhibits Interleukin-12 Secretion by Human Monocytes: an Indirect Interleukin-10-Mediated Effect,” Blood 89(8):2842-8 (1997); Koutsonikolis et al., “HIV-1 Recombinant gp41 Induces IL-10 Expression and Production in Peripheral Blood Monocytes but not in T-Lymphocytes,” Immunol. Lett. 55(2):109-13 (1997); Schols et al., “Human Immunodeficiency Virus Type 1 gp120 Induces Anergy in Human Peripheral Blood Lymphocytes by Inducing Interleukin-10 Production,” J. Virol. 70(8):4953-60 (1996), which are hereby incorporated by reference in their entirety). In animal models, IL-10 has been shown in mice to enhance microbe survival because depletion of IL-10 increased resistance to Candida infection (Tavares et al., “Increased Resistance to Systemic Candidiasis in Athymic or Interleukin-10-Depleted Mice,” J. Infect. Dis. 182(1):266-73 (2000), which is hereby incorporated by reference in its entirety). Moreover, in murine leishmaniasis, a model of Th1 and Th2 immune modulation that is linked to disease outcome, IL-10R blockage by itself can induce near-cure (Murray et al., “Interleukin-10 (IL-10) in Experimental Visceral Leishmaniasis and IL-10 Receptor Blockage as Immunotherapy,” Infect. Immun. 70:6284-6293 (2002), which is hereby incorporated by reference in its entirety). Furthermore, in subsets of patients with active Tb studied, anti-IL-10 Ab has been shown to improve immunity against Mtb, and these patients also have IL-10 producing T cell clones (Gong et al., “Interleukin-10 Downregulates Mycobacterium tuberculosis-Induced Th1 Responses and CTLA-4 Expression,” Infect. Immun. 64(3):913-8 (1996); Mendez-Samperio et al., “Depletion of Endogenous Interleukin-10 Augments Interleukin-1 Beta Secretion by Mycobacterium bovis BCG-Reactive Human Cells,” Clin. Diagn. Lab. Immunol. 4(2):138-41 PMID: 9067646 (1997); Baliko et al., “Th2 Biased Immune Response in Cases with Active Mycobacterium tuberculosis Infection and Tuberculin Anergy,” FEMS Immunol. Med. Microbiol. 22(3):199-204 (1998); Boussiotis et al., “IL-10-Producing T Cells Suppress Immune Responses in Anergic Tuberculosis Patients,” J. Clin. Invest. 105(9): 1317-25 (2000), which are hereby incorporated by reference in their entirety). These data from other infectious agents, coupled with the demonstrated role of IL-10 in modulating tissue bacilli burden and pathology of murine M. bovis infection and the Tb patient data, strongly suggest that IL-10 plays a role in M. tuberculosis infection and disease. The identification of Rv0577 encoding a protein that induces human monocyte production of IL-10 extends the knowledge of one strategy of M. tuberculosis for survival within man that acts by immune suppression.

There is data that shows that only M. tuberculosis subspecies members possess Rv0577 and that Rv0577 is absent from all non-tuberculosis mycobacteria examined thus far. The major subspecies members of Mtb are M. bovis Bacillus Calmette-Guerin (BCG), M. africanum, and M. microti.

In addition, in over 70 M. tuberculosis clinical isolates examined thus far, including representative IS6110 “genotypes”, all possess the Rv0577 gene, and all M. tuberculosis clinical isolates examined thus far express CFP32. Furthermore, CFP32 has been detected in induced sputum of patients with active pulmonary Tb but not from patients with other lung diseases, and importantly, CFP32 levels were positively correlated with IL-10 but not with IFN-γ. These data minimally suggest that Rv0577 is present at the site of human disease, and the association of CFP32 with local IL-10 production is intriguing and implicate a potential role in disease. The finding that the expression of CFP32 (Rv0577) correlates with IL-10 production by human monocytes infected with molecular constructs of M. tuberculosis and M. smegmatis provides evidence for co-opting IL-10 as a mechanism for immunomodulation by M. tuberculosis. This is a potentially new paradigm for M. tuberculosis to evade immune elimination.

### Example 8

### IL-10 Inducing Activity Mapped to Peptides of CFP32

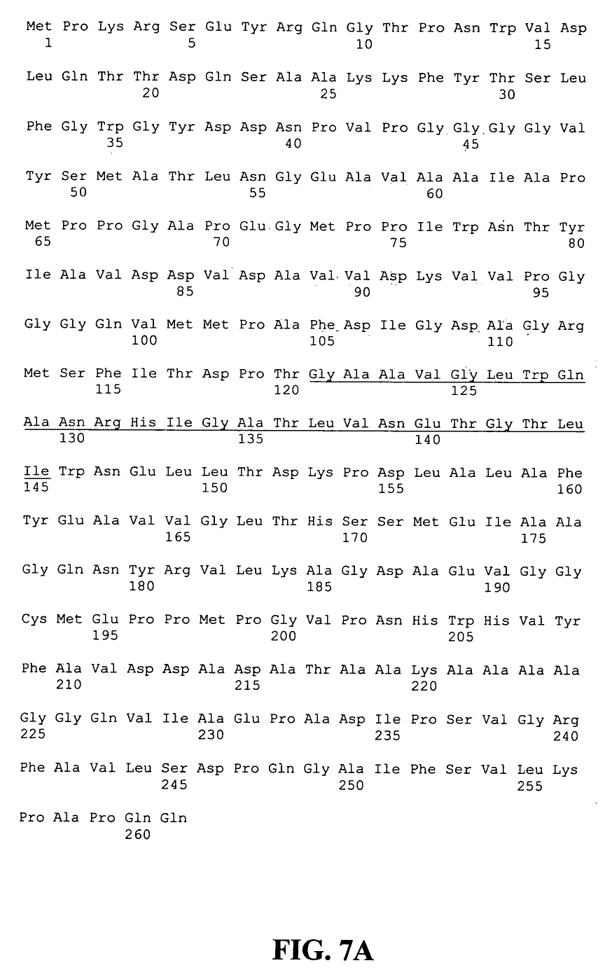

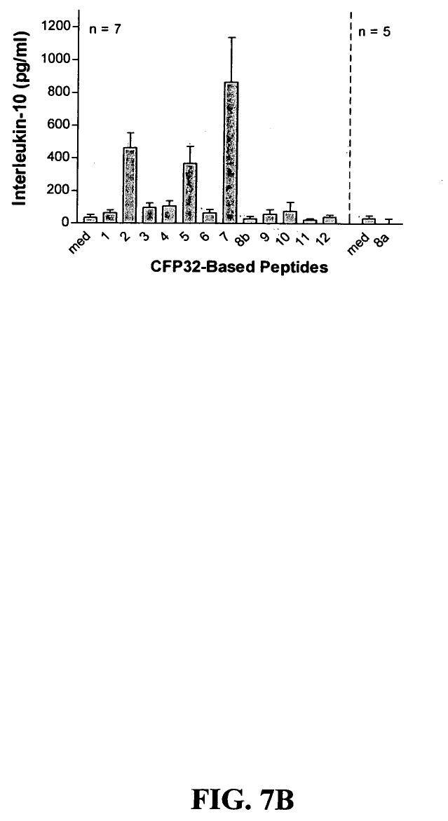

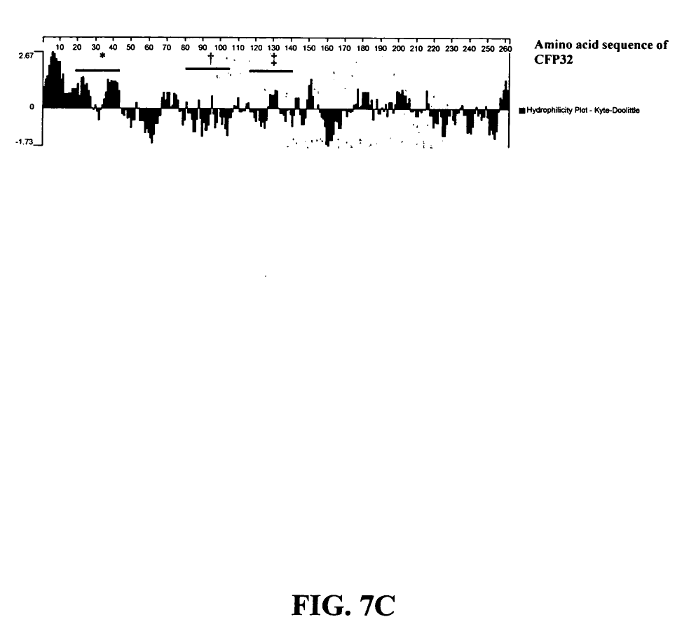

To further evaluate the role of CFP32 in IL-10 induction and map the IL-10 inducing activity of CFP32 to specific CFP32 domains if possible, overlapping peptides (˜25 amino acids each) spanning the full CFP32 protein of 261 amino acids were commercially synthesized (Genosys, Sigma Chemical, St. Louis, Mo.). The amino acids underlined in FIG. 7A shows the peptide fragment (pep7, SEQ ID NO: 44) of the CFP32 amino acid sequence (SEQ ID NO: 2) identified by mass spectroscopy (results shown in FIG. 7C) as the fraction of CFP bearing the highest IL-10 inducing activity. Table 2, above, shows the amino acid sequence of each peptide. Table 3, above, shows the corresponding nucleotide sequence for each CFP32 peptide.

Supernatant harvested from 24 h cultured blood bank donor monocytes stimulated (mostly in triplicates) for 48 h with 40 μg CFP32-based synthetic peptide were assayed for IL-10. Overlapping peptides based on Rv0577 were synthesized commercially (25 aa each for peptides 1 to 11 and 21 aa for peptide 12; each overlapping by 5 aa; peptide 8 design error lead to 5 unrelated aa being introduced (1999 synthesis by Genosys, Sigma Chemical Co., St Louis, Mo.), see Table 1, above for amino acid sequence and residue. FIG. 7B shows that the major IL-10 inducing activity resided in pep7, with pep5 and pep2 having some IL-10 activity and the other peptides showing no activity. Results are the mean and SD of either 5 or 7 donors. Peptides and diluents were found to have <100 pg LPS per 4 μM synthetic peptide by Limulus Amebocyte Lysate (LAL) assay (Sigma, St. Louis, Mo.).

FIG. 7C shows the hydrophilicity plot of CFP32 and peptides-2 (* over bar),−5 († over bar) and −7 (‡ over bar). Peptides 7 and 5 are in a region of relatively high hydrophobicity.

### Example 9

### Immunoreactivity of Antisera Raised Against Rv0577 Whole Recombinant Protein or Peptide Domains

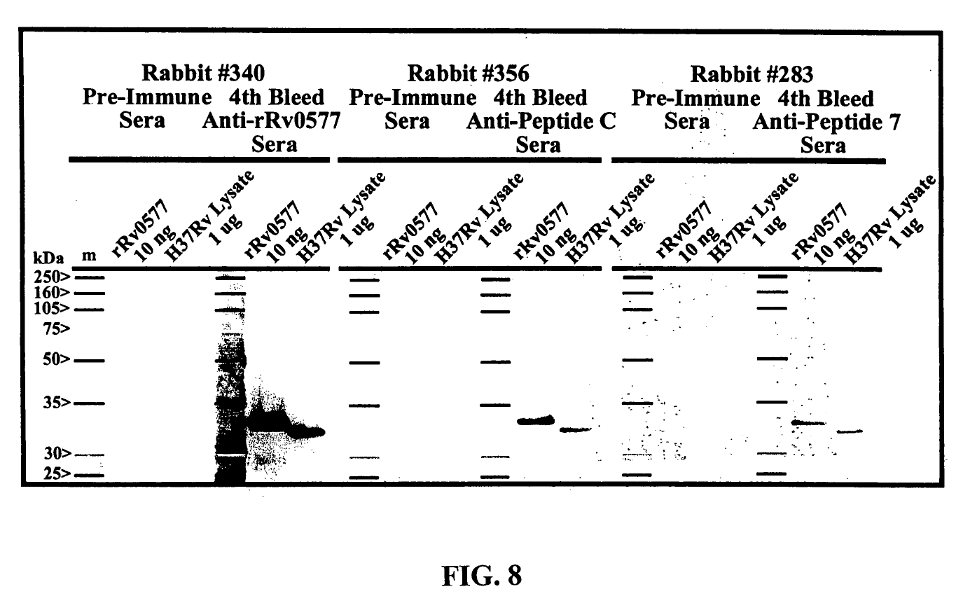

Anti-rRv0577, anti-Peptide C, and anti-peptide 7A each recognized rRv0577 and native Rv0577 in MtbH37Rv lysate. Preimmune serum from each rabbit was universally negative for immune reactivity to rRv0577 and Mtb lysate. Native Rv0577 from MtbH37 lysate and rRv0577 resolved on 15% SDS PAGE transferred to a nitrocellulose membrane were analyzed by WB using preimmune sera or immune sera from rabbits #340 (immunized with whole rRv0577), rabbit #356 (immunized with anti-pep-C) and rabbit #283 (immunized with pep7A). Amounts of pre- and immune sera from each rabbit used were: rabbit #340 (1:1000), rabbit #356 (1:500), and rabbit #283 (1:250). Bound antibodies were visualized by goat anti-rabbit Ab/HRP and chemiluminescence. Results are shown in FIG. 8.

Whole rRv0577 immobilized on polyacrylamide gel or Rv0577 peptides linked to KLH via cysteine were mixed with incomplete Freund's adjuvant to immunize rabbits after a preimmunization bleed (Covance, Research Products, Denver, Pa.). Peptide 7A and peptide C were synthesized at Microchemistry Laboratory, New York Blood Center, New York. Peptide 7A corresponds to amino acid sequences 121-153 of CFP32 SEQ ID NO:2, and includes the pep7 domain (121-145 aa) wherein the major IL-10 inducing activity was found, as described above. Peptide C is the 32 aa C-terminus of CFP32. The amino acid sequences for these CFP32 peptides are as follows:

Pep7A,

(SEQ ID NO: 55)

Acetyl-GAAVGLWQANRHIGATLVNETGTLIWNELLTDK-amide
and
C-peptide,

(SEQ ID NO: 56)

Acetyl-CEPADI PSVGRFAVLSDPQGAIFSVLKPAPQQ-amide

### Example 10

### Subunits Cloned for Expression of Recombinant Rv0577 Proteins

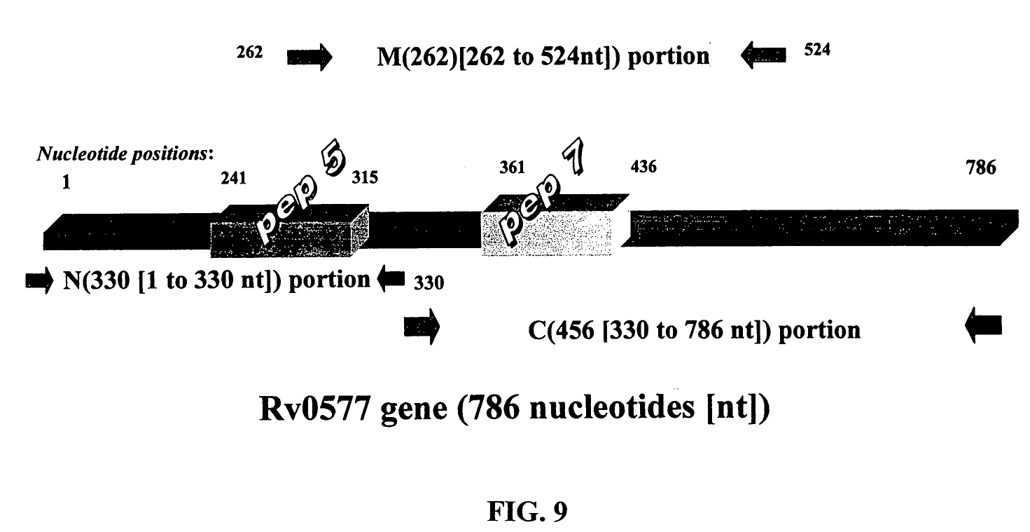

FIG. 9 shows schematically the entire Rv0577 gene with peptide (pep) domains shown to possess IL-10 inducing activity. Pep7 has the major IL-10 inducing activity while pep5 has minor IL-10 inducing activity. As shown FIG. 9, three domains of Rv0577 were cloned: N (330[nt 1 to 330]), M (262[nt 262 to 524]), and C (456[nt 331 to 786]). The primers used for PCR amplification of Mtb H37Rv genomic DNA were as follows:

(SEQ ID NO: 57)

N(330): forward ATAGAATTCGATGCCCAAGAGAAGC;

(SEQ ID NO: 58)
reverse ATTAAGCTTG GCATCGCCGATGTC;
(SEQ ID NO: 59)

C(456): forward ATTAGGATCCGGCCGGATGTCGTTC;

(SEQ ID NO: 60)

reverse ATTAAGCTTTTGCTGCGGTGCGG;
and

(SEQ ID NO: 61)

M(262): forward ATAGGATCCGGCGGTGGTGGACAAGG;

(SEQ ID NO: 62)

reverse ATTAAGCTTAGCTATCTCCATGCTCG.

The expected molecular sizes of the expressed protein from portions of the Rv0577 gene are: N(330), 10 kD; C(456), 15 kD; and M(262), 8.7 kD.

### Example 11

### Expression of Recombinant Rv0577 Subunit Proteins

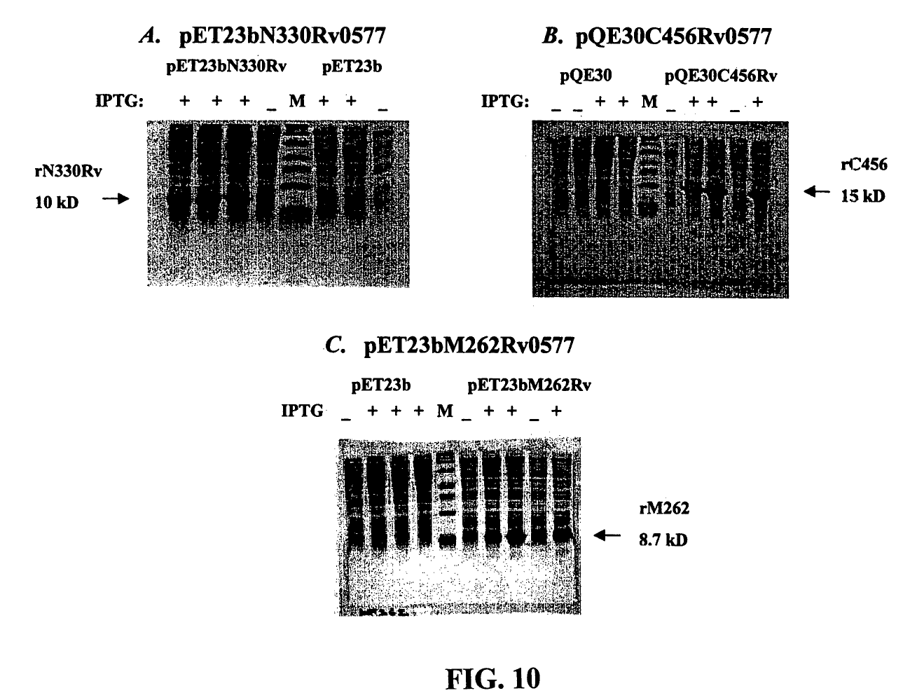

Rv0577 subunit-fragments were amplified from genomic DNA of H37Rv strain of Mtb using the respective primers described above and cloned into expression vectors. The N(330) or M(262) subunits were cloned into the EcoR1 and HindIII restriction sites of the pET23b expression vector. The pET23bN330 or pET23bM262 plasmids were transformed into competent E. coli. DH5α cells and positive clones selected on Luria-agar medium containing ampicillin, chloramphenicol, and tetracycline. Plasmid DNA from the positive clones was isolated and transformed into E coli. BL21 expression host. Protein expression was monitored in Luria broth cultures under IPTG induction (+) or uninduced growth conditions (−). IPTG induced expression of a 10 kD protein from pET23bN330 transformed E. coli, as shown in FIG. 10A, and a 8.7 kD protein from pET23bC262 transformed E. coli, as shown in FIG. 10C. The C(456) subunit was cloned into the BamH1 and HindIII restriction sites of pQE30 expression vector, transformed into E. coli. JM 109 competent cells and positive clones selected on Luria-agar medium containing ampicillin and kanamycin. Plasmid DNA from the positive clones was then isolated and transformed into the M15 E. coli expression host and protein expression monitored under IPTG induction (+) or uninduced growth conditions. FIG. 10B shows IPTG induced (+) expression of a 15 kD protein by E coli transformed with pQE30C456. The negative controls consisting of the plasmid vector only and un-induced growth conditions in E coli transformed with plasmid constructs containing N, C, or M portions of Rv0577 gene are also illustrated in FIG. 10A-C. As shown in FIGS. 10A-C, neither E coli transformed with expression plasmids containing either N-, C- or M-portions of Rv0577 without IPTG induction nor E. coli transformed with expression plasmid only, expressed a protein of the indicated molecular weight. M is the molecular weight marker lane. All cloned DNA were validated by DNA sequence analysis using an automated sequencer (ABI Prism 310 Genetic Analyzer, Perkin Elmer).

### Example 12

### Western Blot Analysis of rCFP32 and Subunits N330, C456

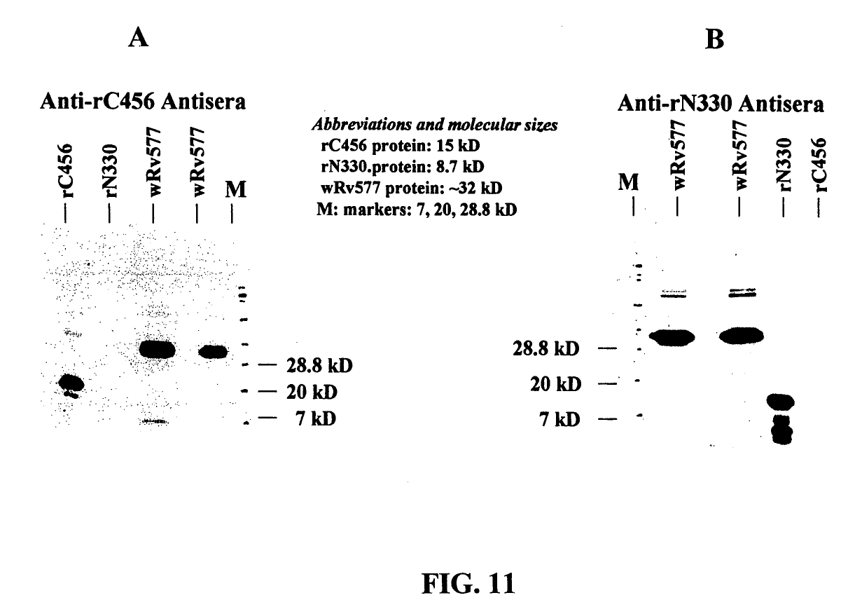

SDS PAGE of the purified recombinant whole protein (rRv0577/CFP32) or subunits N330 and C456 proteins was carried out and transferred to membrane. Western blot (WB) was performed using the murine anti-rC456, shown in FIG. 11A, or anti-rN330 shown in FIG. 11B, Rv0577 subunit sera. FIGS. 11A-B shows that both antisera recognized wRv577. However, anti-C456 recognized only rC456 and not rN330, and anti-N330 recognized rN330 but not rC456 subunit proteins. Murine antisera were raised against the recombinant N330 and C456 Rv0577 subunit His-tagged proteins purified by nickel-agarose column. Mice were immunized subcutaneously on day 1 with soluble protein (1 mg) mixed with adjuvant (Titermax) and booster doses of the same amounts were given subcutaneously on days 10 and 20 followed by one intraperitoneal booster dose on the 30th day. Mice were bled on day 34 day and the serum collected and stored. for later testing of the molecular constructs of Rv0577 to delineate the function and mechanism(s) of action of Rv0577.

### Example 13

### Characterization of Function of CFP32

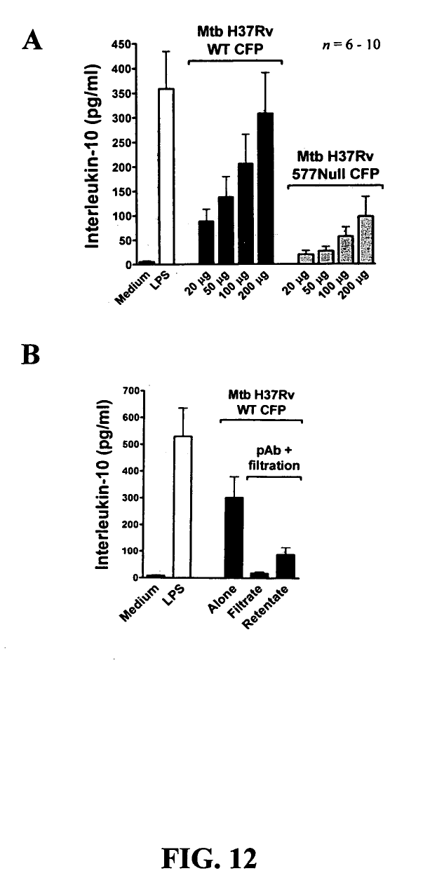

Although studies in which monocytes infected with live WT or mutant Mtb suggested CFP32's role in promoting IL-10 production, CFP32 is a soluble culture filtrate (CF) protein (Huard et al., “The Mycobacterium tuberculosis Complex-Restricted Gene cfp32 Encodes an Expressed Protein That is Detectable in Tuberculosis Patients and Is Positively Correlated with Pulmonary Interleukin-10,” Infection and Immunity 71(12):6871-6883 (2003), which is hereby incorporated by reference in its entirety). Therefore, the ability of CF from WT and 577Null mutant Mtb H37Rv to induce IL-10 in human monocytes was tested. Production of IL-10 triggered by CF from 577Null mutant Mtb was significantly reduced (>60%) when compared to CF from WT Mtb, as seen in FIG. 12A. To further validate that the IL-10 inducing activity in CF is mediated by CFP32, the CF was incubated with rabbit polyclonal sera raised against E. coli expressed rCFP32 (α-rCFP32), and the resulting retentate and filtrate were adjusted to initial volume for testing on monocytes. The rabbit polyclonal antibody weakly neutralized and retained the IL-10 inducing activity as an immune complex in the retentate of a 100 KDa membrane filter, as shown in FIG. 12B. The CFP32-Ab complex in the retentate had residual IL-10 inducing activity because the antisera has weak neutralizing activity. CFP from WT or the 577Null mutant Mtb H37Rv (derived from homologous recombination whereby the Rv0577 gene (cfp32) was replaced by hygromycin gene) were obtained as the retentate using a 10 kDa membrane to concentrate the clarified (by 0.2 μm filter) culture supernatant from exponentially growing cultures previously depleted of bacteria by centrifugation. Freshly isolated monocytes were obtained by negative immune depletion of other cells. Monocytes cultured in X-VIVO20 medium (BioWhittiker, Cambrix Co., Walkersville, Md.) were challenged with agonist for 48 h and IL-10 levels were measured by ELISA (LPS, 100 ng/ml). CFP (100 φg) plus α-rCFP32 (raised against E. coli-CFP32) rabbit antisera (20 μl) incubated for 1 h at room temperature was passed through a 100 KDa column to derive a retentate and filtrate. These data confirm that CFP32 is a Mtb factor with IL-10 inducing activity.

The current work has been directed to further characterize the function of CFP32 and delineate CFP32 mechanism(s) of action. Since 1948, it is well known that virulent strains of Mtb can be distinguished from avirulent strains and saprophytic mycobacteria by their ability to absorb the cationic phenazine neutral red dye (Dubos et al., “Cytochemical Reaction of Virulent Tubercercle Bacilli,” Am Rev Tuberc 58:698-699 (1948), which is hereby incorporated by reference in its entirety). In a recent publication by Andreu et al., the Rv0577 gene was identified as the Mtb “virulence-related neutral red character” through expression of cosmic library of Mtb in M. smegmatis Andreu et al., “Mycobacterium smegmatis Displays the Mycobacterium tuberculosis Virulence-Related Neutral Red Character When Expressing the Rv0577 Gene,” FEMS Microbiol Lett 231:283-289 (2004), which is hereby incorporated by reference in its entirety).

It has been confirmed that M. smegmatis transformed with the pMS3.577 expression plasmid conferred neutral red staining properties. Combined with the data presented here, CFP32 appear to be a multifunctional protein.

### Example 14

### Heterologous Expression of CFP32 in Yeast

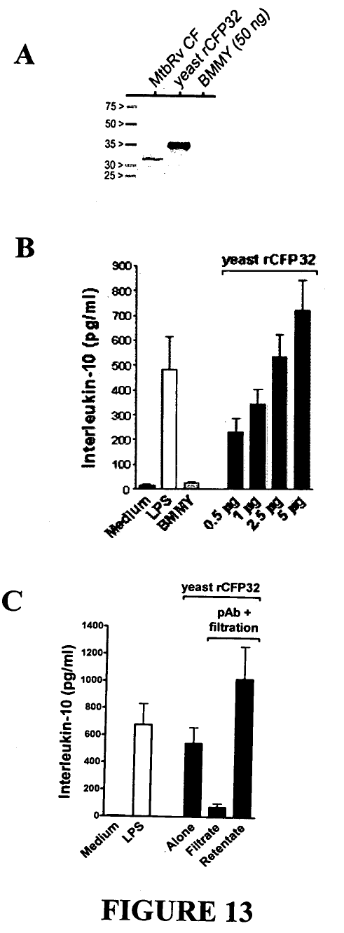

The cfp32 (Rv0577) gene was expressed in yeast, using the rationale that yeast expression may result in an antigen production system more amenable to the heterologous production of functional protein by allowing proper folding and glycosylation of the recombinant protein. Thus, cfp32 was cloned into a modified version of the P. pastoris expression plasmid pPICZ.577 and this construct was introduced into P. pastoris by homologous recombination. A second construct mpPICZ.mis577 containing an unexpressed (out-of-frame) cfp32 insert was used as control. As seen in FIG. 13A, high levels of protein expression were obtained by yeast. The exogenous production of CFP32 by pPICZ.577-transformed yeast is shown in FIG. 13A by western blot carried out using α-rCFP32-E. coli expressed as primary anti-serum, 1 μg of Mtb H37Rv culture filtrate (CF), 10 ng of His-tag purified yeast rCFP32, and 50 ng of concentrated filtered yeast culture medium in which yeast transformed with the missense cfp32 expression vector were grown (BMMY). The difference in size between the native CFP32 and the yeast rCFP32 is due to the presence of the myc-His-tag in the COOH terminal of rCFP32 expressed as a myc-His fusion protein. Yeast specific post-translational modification (acetylation and/or glycosylation) probably additionally contributed to the size difference between yeast rCFP32 and native Mtb CFP32. Similar to CFP32 functional studies in other systems, the histidine (His)-tagged purified yeast rCFP32 specifically induced monocyte IL-10 production in a dose-dependent manner, as shown in FIG. 13B. The P. pastoris-produced rCFP32, as well as BMMY, were used to stimulate at varying doses (in duplicate) fresh peripheral monocytes obtained by negative immune depletion of other cells. Monocytes were cultured in X-VIVO20 medium (BioWhittaker, Cambrix Co., Walkersville, Md.) and the subsequent secretion of TNF-α and IL-10 at 4.5 and 48 h, respectively, was measured by ELISA(LPS, (100 ng/ml). The IL-10-inducing activity is due to the yeast rCFP32 because the anti-sera sequestered the CFP32-antibody complex in the retentate while removing IL-10 inducing activity in the filtrate, as seen in FIG. 13C. Yeast rCFP32 (2.5 μg incubated with 10 μl polyclonal rabbit a-rCFP32 E. coli-expressed) were passed through a 100 kDa membrane generating filtrate and retentate that were subsequently adjusted to original volume. Filtrate (IL-10 removed) and retentate (anti-sera-sequestered CFP32-antibody complex) were used to challenge monocytes and IL-10 levels at 48 h were measured by ELISA. All results are the mean±SD for (n) number of donors performed in duplicate. rCFP32 also promoted TNF-α production in a dose dependent manner, reaching maximal levels at 4-5 h; BMMY show little to no TNF-α-promoting activity. In addition, polymyxin B added to the yeast rCFP32 or CF from WT and mutant Mtb had no effect on IL-10 promotion. A Limulus amebocyte assay showed that the yeast rCFP32 and CF from WT and mutant Mtb are devoid of endotoxin. These data together suggest yeast rCFP32 promotes IL-10 production by monocytes and raise the question whether CFP32 is recognized by monocytes via one or more innate immune receptors.

### Example 15

### Global Antimicrobial Activity is Inhibited by CFP32

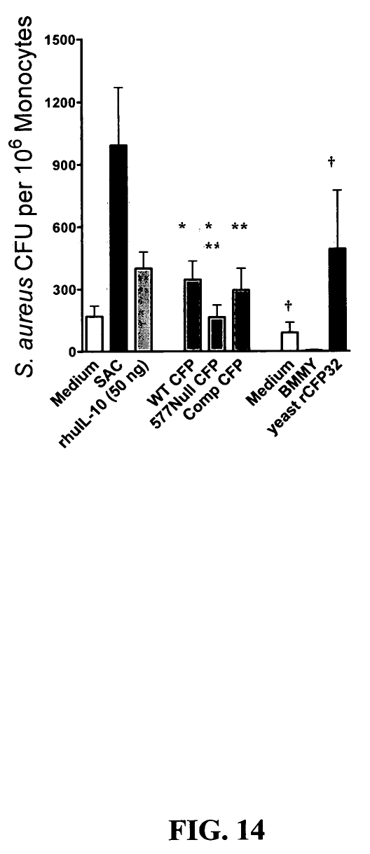

To further characterize the effect of CFP32 on cell function, the killing of an intracellular organism, Staphylococcus aureus, was used as a surrogate for global antimicrobial activity. Antimicrobial function as assayed by survival of intracellular S. aureus was evaluated in monocytes pretreated with yeast rCFP32 at a dose that was mid range in its ability to induce IL-10, culture filtrates proteins (CFP) from Mtb H37Rv wildtype (WT), 577Null mutant and 577complemented 577Null mutant (577Comp or Comp). Human (hu) rIL-10 and heat killed S. aureus cowan strain (SAC) (an agent that triggers TLR2-mediated cell activation and promotes pro-inflammatory cytokine, TNFα, and immunosuppressive cytokine, IL-10), served as positive controls. In this assay system, naive monocytes effectively killed S. aureus, even at the infection ratio of 20 bacteria to one monocyte, shown in FIG. 14. In contrast, killing of S. aureus was suppressed by the pretreatment with rhulL-10 or SAC. CFP from WT and 577Comp mutant Mtb and yeast rCFP32 significantly inhibited the killing of S. aureus. In contrast, CFP from 577Null mutant Mtb and BMMY from yeast transformed with a mis-sense cfp32 gene did not inhibit killing of S. aureus. In brief, fresh monocytes (10⁶ obtained by negative selection) per well in X-VIVO20 medium were treated for 24 h with either medium or SAC (1:1000, Pansorbin), rhuIL-10 (50 ng/ml), 100 μg CFP from Mtb WT, 577Null and 577Comp, 2.5 μg yeast rCFP32, and 2.5 μg BMMY. The medium was removed gently and replaced with pre-warmed 0.5 ml RPMI plus 10% FBS mixed with S. aureus at a ratio of 20 bacteria to monocyte for 2 h 37° C. To kill extracellular bacteria 100 U/ml penicillin and 100 μg/ml streptomycin were added to wells for an additional 2 h. Cells harvested were lysed (0.556% Triton X-100), diluted 10-fold and 100 μl of each sample was plated (in duplicate or triplicate) onto LB agar. Colony counts were enumerated following overnight incubation at 37° C. Illustrated are the mean and SD respectively for n=6, 5, and 12 paired values; P=* 0.005, **0.03, and †0.002, two-tailed t-test.

Confirming CFP32 mediated inhibition of antimicrobial activity is the finding that CF from 577Null mutant Mtb showed no inhibitory activity when compared to medium. In contrast, CF from WT and 577Comp mutant Mtb showed similar promotion of S. aureus survival (inhibitory activity) that was comparable to 50 ng/ml rhulL-10. Of note, thus far, it has not been possible in this assay to neutralize the effect of SAC with a “neutralizing” anti-IL-10R mAb. This finding may imply that the inhibition of antimicrobial killing may be more than just IL-10 induction. Based on the finding that CFP32 promoted TNFα and IL-6 in addition to L-10 expression, it is likely that the inhibition of intracellular killing of S. aureus is via several mechanisms that act in sum to disarm monocytes' antimicrobial activity despite the primary activating activity of TNFα. Detailing further the effect of rCFP32 on immune function in vitro and in vivo, and genes that are up-regulated after exposure to CFP32, as well as how host innate immune cells are activated by CFP32 may provide information for the commercial application of whole molecule or a subunit domain of CFP32.

### Example 16

### rCFP32 Activates Innate Immune Cells via TLR2

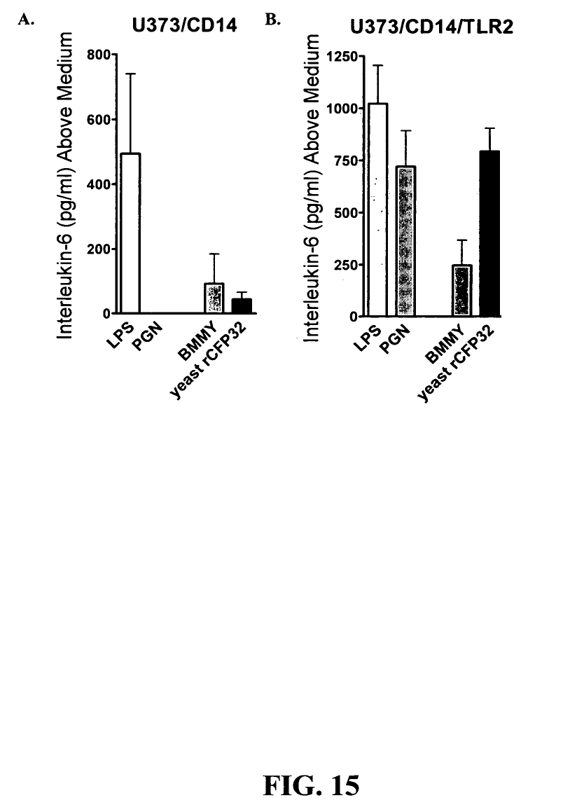

Toll like receptors (TLR) are a family of pattern recognition receptors, which recognize classes of microbial ligands and signal to activate innate immune cells. Studies have shown that TLR4 and TLR2 activation rapidly triggers the production of pro-inflammatory cytokines, and the immunosuppressive cytokine, IL-10. Studies of Mtb infection using TLR4 or TLR2 null mice have shown that TLR2 is required for immunity against Mtb challenge Drennan et al., “Toll-Like Receptor 2-Deficient Mice Succumb to Mycobacterium tuberculosis Infection,” Am J Pathol 164(1):49-57 (2004); Shim et al., “Toll-Like Receptor 4 Plays No Role in Susceptibility of Mice to Mycobacterium tuberculosis Infection,” Tuberculosis (Edinb) 83(6):367-371 (2003); which are hereby incorporated by reference in their entirety). In light of CFP32's IL-10 inducing activity, it was evaluated whether TLR4 or TLR2 may be involved in innate immune cell activation. Experiments were performed using the human U373 glioma (neural macrophage) cell line. The U373 cells were stably transfected with human CD14 (U373/CD14) and a clonal sub-lineage was subsequently established by stable transfection of TLR2 (U373/CD14/TLR2). U373 cells express TLR4 and are LPS-responsive but do not normally express CD14 nor TLR2. rCFP32 expressed by the yeast, P. pastoris, and BMMY (medium from yeast transformed with plasmid having an out-of-frame CFP32 insert) were tested in macrophage-like (glioma) cell lines, U373/CD14, shown in FIG. 15A, and U373/CD14/TLR2, shown in FIG. 15B. Yeast rCFP32 (2.5 μg/ml) similar to peptidoglycan (15 μg/ml, PGN) activated U373/CD14/TLR2 cells but not U373/CD14 cells. In contrast, 100 ng/ml LPS potently activated both cell lines; while BMMY weakly activity in both cell lines. U373 cells express TLR4 and are LPS-responsive but do not normally express CD14 nor TLR2. In brief, U373 cells were transfected with pCEP4/CD14 by the calcium phosphate method similar to as previously described (Delude et al., “Construction of a Lipopolysaccharide Reporter Cell Line and its Use in Identifying Mutants Defective in Endotoxin, but not TNF-alpha, Signal Transduction,” J Immunol 161(6):3001-3009 (1998), which is hereby incorporated by reference in its entirety). The bulk transfected cells were sorted for CD14 expression by FACS and cloned by limiting dilution to derive a stably transfected U373/CD14 cell line. This cell line was further transfected by the calcium phosphate method with the vector pFLAG containing the cDNA of human TLR2 as previously described (Yoshimura et al., “Cutting Edge: Recognition of Gram-Positive Bacterial Cell Wall Components by the Innate Immune System Occurs via Toll-Like Receptor 2,” J Immunol 163(l):1-5 (1999), which is hereby incorporated by reference in its entirety). The bulk transfected cells were sorted by FACS for CD14 and TLR2 expression and cloned by limiting dilution to derive the stably transfected U373/CD14/TLR2 cell line. The U373 cell lines were maintained in DMEM medium containing 10% FBS, L-glutamine, penicillin, and streptomycin (Lien et al., “A Novel Synthetic Acyclic Lipid A-Like Agonist Activates Cells Via the Lipopolysaccharide/Toll-Like Receptor 4 Signaling Pathway,” J Biol Chem 276(3):1873-1880 (2001), which is hereby incorporated by reference in its entirety). FACS analysis confirmed that both clonal cell lines express CD14 and that only U373/CD14/TLR2 possessed surface TLR2. As a control, culture supernatant (BMMY) from yeast transformed with a vector carrying missense CFP32 when tested at the same protein concentration as that of rCFP32 induced the production of a small amount of IL-6 in both U373/CD 14 and U373/CD14/TLR2 cell lines. These findings confirm CFP32 as the ligand triggering TLR2-mediated cell activation, and argue against the activation activity solely due to yeast contaminants co-purified with His-tagged rcFP32.

Currently it is not known precisely how TLRs recognize diverse arrays of molecules especially when, in many cases, the molecules are not polypeptides. One theory advanced is that TLRs interact with specific hypophobic regions of target molecules. Therefore, CFP32 was analyzed for hydrophobic domains. Of interest is the finding that regions occupied by peptide 7 and peptide 5 are of relatively high hydrophobicity, as shown in FIG. 7C, and thereby may account for their ability to bind to TLR2, and trigger cell activation signaling.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of treating an inflammatory condition in a mammal comprising:
providing a polypeptide comprising either: 1) the amino acid sequence of SEQ ID NO: 34; 2) the amino acid sequence of SEQ ID NO: 40; or 3) the amino acid sequence of SEQ ID NO: 44; and
administering the polypeptide to a mammal under conditions effective to treat the inflammatory condition.

2. The method according to claim 1, wherein the polypeptide comprises the amino acid sequence of SEQ ID NO: 34.

3. The method according to claim 1, wherein the polypeptide comprises the amino acid sequence of SEQ ID NO: 40.

4. The method according to claim 1, wherein the polypeptide comprises the amino acid sequence of SEQ ID NO: 44.

5. The method according to claim 1, wherein the inflammatory condition is an autoimmune disease.

6. The method according to claim 5, wherein the autoimmune disease is selected from the group consisting of lupus, rheumatoid arthritis, autoimmune arthritis, scleroderma, inflammatory bowel condition, multiple sclerosis, polyarteritis nodosum, and temporal arteritis.

7. The method according to claim 1, wherein the inflammatory condition is selected from the group consisting of bronchiectasis, asthma, sepsis, and tropical spastic paralysis caused by HTLV-1.

8. A method of treating an inflammatory condition in a mammal comprising:
providing a nucleic acid construct comprising:
a nucleic acid comprising either: 1) the nucleotide sequence of SEQ ID NO: 33; 2) the nucleotide sequence of SEQ ID NO: 39; 3) the nucleotide sequence of SEQ ID NO: 43; 4) a nucleic acid molecule encoding a polypeptide comprising the amino acid sequence of SEQ ID NO: 34; 5) a nucleic acid molecule encoding a polypeptide comprising the amino acid sequence of SEQ ID NO: 40; or 6) a nucleic acid molecule encoding a polypeptide comprising the amino acid sequence of SEQ ID NO: 44;
an operably linked DNA promoter; and
an operably linked 3′ regulatory region; and
administering the nucleic acid construct to a mammal under conditions effective to treat the inflammatory condition.

9. The method according to claim 8, wherein the nucleic acid molecule comprises the nucleotide sequence of SEQ ID NO: 33.

10. The method according to claim 8, wherein the nucleic acid molecule comprises the nucleotide sequence of SEQ ID NO: 39.

11. The method according to claim 8, wherein the nucleic acid molecule comprises the nucleotide sequence of SEQ ID NO: 43.

12. The method according to claim 8, wherein the inflammatory condition is an autoimmune disease.

13. The method according to claim 12, wherein the autoimmune disease is selected from the group consisting of lupus, rheumatoid arthritis, autoimmune arthritis, scleroderma, inflammatory bowel condition, multiple sclerosis, polyarteritis nodosum, and temporal arteritis.

14. The method according to claim 8, wherein the inflammatory condition is selected from the group consisting of bronchiectasis, asthma, sepsis, and tropical spastic paralysis caused by HTLV-1.

15. A method of vaccinating a mammal against infection by Mycobacterium tuberculosis comprising:
administering to the mammal an effective amount of an isolated polypeptide, wherein the polypeptide comprises either: 1) the amino acid sequence of SEQ ID NO: 34; 2) the amino acid sequence of SEQ ID NO: 40; or 3) the amino acid sequence of SEQ ID NO: 44.

16. The method according to claim 15, wherein said administering is oral, intradermal, intramuscular, intraperitoneal, intravenous, subcutaneous, or intranasal.

17. A vaccine for preventing Mycobacterium tuberculosis infection and disease in mammals comprising:
an isolated polypeptide comprising either: 1) the amino acid sequence of SEQ ID NO: 34; 2) the amino acid sequence of SEQ ID NO: 40; or 3) the amino acid sequence of SEQ ID NO: 44.

18. The vaccine according to claim 17, wherein said polypeptide is purified.

19. A method of vaccinating mammals against infection by Mycobacterium tuberculosis comprising:
administering an effective amount of the vaccine according to claim 17 to mammals.

20. The method according claim 19, wherein said administering is oral, intradermal, intramuscular, intraperitoneal, intravenous, subcutaneous, or intranasal.

21. A method of treating mammals infected with Mycobacterium tuberculosis comprising:
administering an effective amount of the antibody or binding portion thereof raised against a polypeptide comprising either: 1) the amino acid sequence of SEQ ID NO: 34; 2) the amino acid sequence of SEQ ID NO: 40; or 3) the amino acid sequence of SEQ ID NO: 44 to mammals infected with Mycobacterium tuberculosis, thereby treating the infection.

22. The method according to claim 21, wherein said administering is oral, intradermal, intramuscular, intraperitoneal, intravenous, subcutaneous, or intranasal.

23. The method according to claim 21, wherein a monoclonal or polyclonal antibody is administered.

24. The method according to claim 21, wherein a binding portion selected from the group consisting of an Fab fragment, an F(ab′)₂ fragment, and an Fv fragment, is administered.

25. A method of enhancing vaccination against Mycobacterium tuberculosis comprising:
suppressing in the microorganism the expression of a nucleic acid molecule comprising either: 1) the nucleotide sequence of SEQ ID NO: 33; 2) the nucleotide sequence of SEQ ID NO: 39; 3) the nucleotide sequence of SEQ ID NO: 43; 4) a nucleic acid molecule encoding a polypeptide comprising the amino acid sequence of SEQ ID NO: 34; 5) a nucleic acid molecule encoding a polypeptide comprising the amino acid sequence of SEQ ID NO: 40; or 6) a nucleic acid molecule encoding a polypeptide comprising the amino acid sequence of SEQ ID NO: 44 and
administering the suppressed microorganism to a subject under conditions effective to enhance vaccination against Mycobacterium tuberculosis.

26. The method according to claim 25, wherein the microorganism is Mycobacterium bovis Bacillus Calmette-Guerin.
